(19) United States
(12) Patent Application Publication
(10) Pub. No.: US 2007/0031963 A1
(43) Pub. Date: Feb. 8, 2007

(54) Cell culture flasks, systems, and methods for automated processing

(76) Inventors: Jim Yuchen Chang, San Diego, CA (US); James Keven Mainquist, San Diego, CA (US); Robert Charles Downs, La Jolla, CA (US); Randal Joseph Wayne, San Diego, CA (US); Daniel Glen Sipes, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/444,131

(22) Filed: May 31, 2006

Related U.S. Application Data
(60) Provisional application No. 60/686,753, filed on Jun. 1, 2005.

(51) Int. Cl. C12M 1/24; G01N 9/30

(52) U.S. Cl. 435/304.3; 435/304.2; 422/72; 422/102

(57) ABSTRACT

Cell culture flasks that are readily conducive to various automated processing applications, including introducing and removing fluid from the flasks are provided. Automated cell culture flask processing systems, system components, and related methods are also provided.

### RELATED APPLICATIONS

This application claims priority to U.S. Provisional Application No. 60/686,753, filed Jun. 1, 2005, the disclosure of which is hereby incorporated by reference in its entirety.

### BACKGROUND OF THE INVENTION

1. Field of the Invention

The present invention relates generally to laboratory ware and instrumentation, such as cell culture flasks and related systems, components, and methods.

2. Description of the Related Art

Cells and tissues are commonly cultured in vitro in various types of cell culture containers or flasks. The cells, or by-products (e.g., proteins, nucleic acids, metabolites, etc.) of the cells cultivated in such flasks, are used in assorted disciplines related to biotechnology, including medicine, pharmacology, and genetic research and engineering.

To enhance throughput, many aspects of biotechnology are becoming increasingly automated. However, many pre-existing cell culture flasks are not conducive to automated processing applications. Accordingly, there exists a need for cell culture flasks that can be accessed efficiently or otherwise processed using automated systems. The present invention fulfills these and other needs.

### SUMMARY OF THE INVENTION

Embodiments of the present invention provide various cell culture flasks that are readily conducive to automated processing, including introducing and removing fluids from the flasks using automated fluid handling systems. For example, the invention provides a cell culture flask having dimensions that correspond to those of a standard multi-well or microtiter plate. In addition, many of these flasks can be accessed while positioned in a horizontal position, e.g., in a nest of positioning components of an automated system. The invention also provides related systems, components, and methods.

In one aspect, the invention provides a cell culture flask that includes a culture chamber that is formed by a bottom wall, a top wall, a first side wall and a second side wall that is opposite to the first side wall, a first end wall and a second end wall that is opposite to the first end wall. The cell culture flask also includes a vent opening in the top wall that allows air exchange between the culture chamber and an exterior of the flask, and an access port opening in the top wall through which fluids can be introduced into or removed from the culture chamber. Typically, the vent opening comprises a filter. In some embodiments, the vent opening extends (e.g., at least about 5 mm, at least about 10 mm, at least about 25 mm, at least about 50 mm, etc.) from an external surface of the cell culture flask. In addition, the cell culture flask is configured to allow introduction or removal of fluid through the access port opening into the culture chamber when the cell culture flask is positioned in a horizontal position. Optionally, the cell culture flask includes dimensions that substantially correspond to dimensions of a standard multi-well plate. In some embodiments, at least one tier is formed within the culture chamber.

In certain embodiments, the cell culture flask includes at least one locational feature (e.g., the vent opening, the access port opening, and/or a label or other feature of the flask) that is positioned to trigger a sensor (e.g., a positioning laser sensor, etc.) of a storage device when the cell culture flask is stored in the storage device.

In addition, various types of labeling features, e.g., bar codes and the like, are optionally associated (e.g., disposed on flask surfaces, fabricated integral with the flasks, etc.) with the cell culture flasks described herein. A labeling feature typically provides information about the particular flask, such as its identity, contents, creation date, location, movement dates, activity dates, destination, etc. To further illustrate, bar codes are optionally added to any wall of the flasks. In some embodiments, for example, the flasks have bar codes on the exterior of both end walls so that a bar code reader on a robotic gripping device can read it while handling the flask. Two or more bar codes are generally used for multi-robot cell or work perimeter access. In these embodiments, the robots can hand off the flasks without having to rotate the flask with, e.g., two bar codes. In some of these embodiments, the bar codes are set to be even on one end and odd on the other end.

In some embodiments, the cell culture flask includes a cell concentration cavity disposed in at least one wall of the culture chamber. The cell concentration cavity is generally disposed in the wall of the culture chamber at a position such that the cell concentration cavity is above a selected fluid volume when the selected fluid volume is contained in the culture chamber. The cell concentration cavity is structured to concentrate cells from a fluid medium contained in the culture chamber when the cell culture flask is subjected to a sufficient applied centrifugal force (e.g., about 1000 g, etc.). The cell concentration cavity typically has a cross-sectional shape selected from, e.g., a regular n-sided polygon, an irregular n-sided polygon, a triangle, a square, a rectangle, a trapezoid, a circle, an oval, and the like. Typically, one or more walls of the culture chamber slope towards the cell concentration cavity, e.g., to funnel cells into the cavity under an applied centrifugal force.

In certain embodiments, the top wall comprises a baffle that communicates with the vent opening. The baffle is structured to prevent fluid from entering the vent opening when the fluid contacts the baffle. In some of these embodiments, the baffle comprises a shield having one or more holes disposed through the shield. The holes are typically sized such that surface tension of the fluid causes one or more bubbles to form when the fluid contact the shield to prevent fluid from entering the vent opening. In other exemplary embodiments, the baffle comprises one or more channels that direct the fluid away from the vent opening when the fluid enters the baffle.

Typically, the cell culture flask includes a closure (e.g., a septum, a lid, etc.) that closes the access port opening. In some embodiments, the closure comprises a material exchange region and a contour of the closure is shaped to direct fluid away from the material exchange region when the fluid contacts the material exchange region. In these embodiments, the contour of the closure disposed proximal to the material exchange region is typically rounded. The material exchange region generally includes a self-sealing channel disposed through the closure (e.g., a pre-pierced septum, etc.).

In another aspect, the invention provides a cell culture flask that includes a culture chamber that is formed by a bottom wall, a top wall, a first side wall and a second side wall that is opposite to the first side wall, a first end wall and a second end wall that is opposite to the first end wall. The cell culture flask also includes a cell concentration cavity disposed in at least one wall of the culture chamber. The cell concentration cavity is structured to concentrate cells from a fluid medium contained in the culture chamber when the cell culture flask is subjected to a sufficient applied centrifugal force. Typically, the cell concentration cavity has a cross-sectional shape selected from, e.g., a regular n-sided polygon, an irregular n-sided polygon, a triangle, a square, a rectangle, a trapezoid, a circle, an oval, and the like. In some embodiments, one or more walls of the culture chamber slope towards the cell concentration cavity. The cell concentration cavity is typically disposed in the wall of the culture chamber at a position such that the cell concentration cavity is above a selected fluid volume when the selected fluid volume is contained in the culture chamber.

In another aspect, the invention provides a cell culture flask that includes a culture chamber that is formed by a bottom wall, a top wall, a first side wall and a second side wall that is opposite to the first side wall, a first end wall and a second end wall that is opposite to the first end wall. The cell culture flask also includes a vent opening in at least one wall of the culture chamber that allows air exchange between the culture chamber and the exterior of the flask. In addition, the cell culture flask also includes a baffle that communicates with the vent opening. The baffle is structured to prevent fluid from entering the vent opening when the fluid contacts the baffle. In some embodiments, for example, the baffle comprises a shield having one or more holes disposed through the shield. The holes are sized such that surface tension of the fluid causes one or more bubbles to form when the fluid contact the shield to prevent fluid from entering the vent opening. In certain embodiments, the baffle comprises one or more channels that direct the fluid away from the vent opening when the fluid enters the baffle.

In another aspect, the invention provides a cell culture flask that includes a culture chamber that is formed by a bottom wall, a top wall, a first side wall and a second side wall that is opposite to the first side wall, a first end wall and a second end wall that is opposite to the first end wall. The cell culture flask also includes an access port opening in at least one wall of the culture chamber through which fluids can be introduced into or removed from the culture chamber. In addition, the cell culture flask also includes a closure (e.g., a septum, a lid, etc.) that closes the access port opening. The closure comprises a material exchange region in which a contour of the closure is shaped to direct fluid away from the material exchange region when the fluid contacts the material exchange region. The contour of the closure disposed proximal to the material exchange region is typically rounded. Optionally, the material exchange region comprises a self-sealing channel disposed through the closure.

In another aspect, the invention provides a cell culture flask that includes a culture chamber that is formed by a bottom wall, a top wall, a first side wall and a second side wall that is opposite to the first side wall, a first end wall and a second end wall that is opposite to the first end wall. The cell culture flask also includes a vent opening in at least one wall of the culture chamber and extending (e.g., at least about 5 mm, at least about 10 mm, at least about 25 mm, at least about 50 mm, etc.) from an external surface of the cell culture flask. The vent opening allows air exchange between the culture chamber and an exterior of the flask.

In another aspect, the invention provides a container closure that includes a material exchange region in which a contour of the container closure is shaped to direct fluid away from the material exchange region when the fluid contacts the material exchange region. In some embodiments, the contour of the closure disposed proximal to the material exchange region is rounded. Optionally, the material exchange region comprises a self-sealing channel disposed through the closure.

In another aspect, the invention provides a cell culture flask processing system. The system includes a processing head, a cell culture flask positioning component that is structured to position at least one cell culture flask in a horizontal position, and a translational mechanism operably connected to the processing head and/or the cell culture flask positioning component. The translational mechanism is configured to move the processing head and/or the cell culture flask positioning component relative to one another such that the processing head communicates with the cell culture flask when the cell culture flask positioning component positions the cell culture flask in the horizontal position. Typically, the cell culture flask processing system includes a controller operably connected to the processing head, the cell culture flask positioning component, and/or the translational mechanism.

In some embodiments, the processing head includes at least one tip, and in which the translational mechanism is configured to move the processing head and/or the cell culture flask positioning component relative to one another such that the tip accesses the cell culture flask when the cell culture flask positioning component positions the cell culture flask in a horizontal position. In certain embodiments, the processing head comprises multiple tips that are configured to access multiple cell culture flasks when the cell culture flasks are stacked relative to one another on the cell culture flask positioning component. In some of these embodiments, the cell culture flask processing system includes a fluid conveyance mechanism operably connected to the tip. The fluid conveyance mechanism is configured to introduce fluid into and/or remove fluid from the cell culture flask through the tip when the tip accesses the cell culture flask. In certain embodiments, the processing head includes at least two tips, and the fluid conveyance mechanism is configured to recirculate fluid disposed in the cell culture flask through the tips when the tips access the cell culture flask.

In some embodiments, the tip is configured to allow gas exchange between the cell culture flask and an exterior of the cell culture flask when the tip accesses the cell culture flask. In these embodiments, a filter is typically operably connected to the tip.

In certain embodiments, the processing head comprises a pressure head configured to communicate with a vent opening of the cell culture flask. In these embodiments, the cell culture flask processing system generally includes a pressure source operably connected to the pressure head. The pressure source is typically configured to apply pressure to the pressure head when the pressure head communicates with the vent opening of the cell culture flask to effect displacement of fluid from the vent opening of the cell culture flask.

In another aspect, the invention provides a centrifuge rotor that includes at least one nest that is structured to receive a cell culture flask. The centrifuge rotor is structured to rotate the cell culture flask in a horizontal position so that cells in a cell suspension contained in the cell culture flask collect on a side wall of the cell culture flask. In certain embodiments, the nest is configured to associate with a lift mechanism such that the lift mechanism can raise and/or lower the cell culture flask when the cell culture flask is present in the nest and the centrifuge rotor is at rest. In some of these embodiments, for example, an orifice is disposed through the nest. The orifice allows the lift mechanism to raise and/or lower the cell culture flask when the cell culture flask is present in the nest and the centrifuge rotor is at rest.

In still another aspect, the invention provides a centrifuge rotor that includes at least one nest that is structured to receive a cell culture flask. The nest is configured to associate with a lift mechanism such that the lift mechanism can raise and/or lower the cell culture flask when the cell culture flask is present in the nest and the centrifuge rotor is at rest. In some embodiments, for example, the centrifuge rotor includes an orifice disposed through the nest. The orifice allows the lift mechanism to raise and/or lower the cell culture flask when the cell culture flask is present in the nest and the centrifuge rotor is at rest.

The centrifuge rotors described herein include various embodiments. For example, a position of the nest is typically fixed in the centrifuge rotor. Optionally, the nest comprises one or more angled surfaces that direct the cell culture flask into the nest when the nest receives the cell culture flask. Typically, the nest comprises one or more retaining features that retain the cell culture flask when the centrifuge rotor rotates. In some embodiments, the centrifuge rotor includes one or more pivotal positioning components that are structured to receive the cell culture flask or another container. The pivotal positioning components pivot when the centrifuge rotor rotates. The invention also provides centrifuge systems that include the centrifuge rotors described herein.

In another aspect, the invention provides a method of processing a cell culture flask. The method includes positioning the cell culture flask in a horizontal position. In addition, the method also includes introducing and/or removing fluid through an access port opening disposed in a top wall of the cell culture flask to thereby process the cell culture flask.

In another aspect, the invention provides a method of concentrating cells in a cell culture flask. The method includes placing a cell culture flask containing a cell suspension into a centrifuge rotor. The method also includes rotating the cell culture flask in a horizontal position in the centrifuge rotor at a rate that is sufficient to cause cells in the cell suspension to collect on a side wall of the cell culture flask to thereby concentrate the cells in the cell culture flask.

### BRIEF DESCRIPTION OF THE DRAWINGS

FIG. 1A schematically shows a cell culture flask from a perspective view according to one embodiment of the invention.

FIG. 1B schematically illustrates the cell culture flask of FIG. 1A from a side elevational view.

FIG. 1C schematically shows a cell culture flask that includes multiple tiers according to one embodiment of the invention.

FIG. 2A schematically depicts a segment of cell culture flask wall having a closure disposed in an access port opening according to one embodiment of the invention.

FIG. 2B schematically depicts another segment of a cell culture flask wall having an embodiment of a septum disposed in an access port opening.

FIG. 2C schematically depicts another segment of a cell culture flask wall having an alternate embodiment of a septum disposed in an access port opening.

FIG. 2D schematically depicts another segment of a cell culture flask wall having yet another alternate embodiment of a septum disposed in an access port opening.

FIG. 2E schematically depicts another segment of a cell culture flask wall having an embodiment of a retention feature configured to retain a septum.

FIG. 2F schematically depicts another segment of a cell culture flask wall having another embodiment of a retention feature configured to retain a septum.

FIG. 2G schematically depicts another segment of a cell culture flask wall having another embodiment of a retention feature configured to retain a septum.

FIGS. 3A-D schematically show cell culture flasks having different vent and access port opening configurations according to various embodiments of the invention.

FIGS. 4A-C schematically show cell culture flasks having different volume capacities according to various embodiments of the invention.

FIGS. 5A and B schematically illustrate cutaway, cross-sectional views of a cell culture flask having a septum positioned to facilitate fluid removal from the flask according to one embodiment of the invention.

FIG. 5C schematically illustrates a perspective view of a cell culture flask having a septum shaped to facilitate fluid removal from the flask according to one embodiment of the invention.

FIG. 6A schematically shows a cutaway perspective view of a cell culture flask that includes a cell concentration cavity according to one embodiment of the invention.

FIG. 6B schematically illustrates the cell culture flask of FIG. 6A from a cross-sectional side view.

FIG. 6C schematically illustrates the cell culture flask of FIG. 6A from a top view

FIG. 7 schematically shows a cross-sectional view of a cell culture flask that includes a cell concentration cavity according to one embodiment of the invention.

FIG. 8A schematically shows a cell culture flask having a cell concentration cavity from a cross-sectional side view according to one embodiment of the invention.

FIG. 8B schematically shows the cell culture flask of FIG. 8A from a cross-sectional top view.

FIG. 9 schematically shows a cell culture flask having a cell concentration cavity from a cross-sectional side view according to one embodiment of the invention.

FIG. 10 schematically shows a cell culture flask having a vent opening that extends from an external surface of a cell culture flask according to one embodiment of the invention.

FIG. 11A schematically shows a cross-sectional side view of cell culture flask that includes a baffle according to one embodiment of the invention.

FIG. 11B schematically illustrates a detailed bottom view of the baffle from FIG. 11A.

FIG. 12 schematically shows a cross-sectional side view of cell culture flask that includes a baffle according to one embodiment of the invention.

FIG. 13 schematically depicts a cross-sectional view of a processing head positioned to access stacked cell culture flasks according to one embodiment of the invention.

FIG. 14 schematically shows a cross-sectional view of a cell culture flask configured for the recirculation of fluids in the flask according to one embodiment of the invention.

FIG. 15 schematically illustrates a cross-sectional view of a cell culture flask having a venting tip according to one embodiment of the invention.

FIG. 16 schematically depicts a cross-sectional view of a pressure head communicating with a vent opening of a cell culture flask according to one embodiment of the invention.

FIG. 17 schematically depicts an exemplary cell culture flask processing system according to one embodiment of the invention.

FIG. 18A schematically shows a cross-sectional side view a centrifuge rotor according to one embodiment of the invention.

FIG. 18B schematically shows a top view of the centrifuge rotor of FIG. 18A.

FIG. 18C schematically illustrates a cross-sectional view of a nest from the centrifuge rotor of FIG. 18A.

FIG. 18D schematically illustrates a cross-sectional view of a nest from the centrifuge rotor of FIG. 18A further showing a cell culture flask raised by a lift mechanism from the nest.

FIG. 19 schematically shows a detailed cross-sectional, cutaway view of a retaining feature of a centrifuge rotor according to one embodiment of the invention.

FIG. 20 schematically shows a top view of a centrifuge rotor that includes pivotal positioning components according to one embodiment of the invention.

### DETAILED DESCRIPTION

Definitions

Before describing the present invention in detail, it is to be understood that this invention is not limited to particular embodiments. It is also to be understood that the terminology used herein is for the purpose of describing particular embodiments only, and is not intended to be limiting. As used in this specification and the appended claims, the singular forms “a,” “an,” and “the” also include plural referents unless the context clearly provides otherwise. Thus, for example, reference to “a vent opening” also includes more than one vent opening. Units, prefixes, and symbols are denoted in the forms suggested by the International System of Units (SI), unless specified otherwise. Numeric ranges are inclusive of the numbers defining the range. Further, unless defined otherwise, all technical and scientific terms used herein have the same meaning as commonly understood by one of ordinary skill in the art to which the invention pertains. The terms defined below, and grammatical variants thereof, are more fully defined by reference to the specification in its entirety.

The term “automated” refers to a process, device, sub-system, or system that is controlled at least in part by mechanical and/or electronic devices in lieu of direct human control. In certain embodiments, for example, the cell culture flasks of the invention are processed in systems in the absence of direct human control.

The term “bottom” refers to the lowest point, level, surface, or part of a device or system, or device or system component, when oriented for typical designed or intended operational use.

Device or system components “communicate” with one another when fluids, energy, pressure, information, objects, or other matter can be transferred between those components.

The term “fluid” refers to matter in the form of gases, liquids, semi-liquids, pastes, or combinations of these physical states. Exemplary fluids include certain reagents for performing a given assay, various types of media for supporting a cell culturing process, suspensions of cells, beads, or other particles, and/or the like.

The term “horizontal” refers to a plane that is approximately parallel to a plane of a supporting surface.

The term “standard” in the context of microtiter plates refers to standards for microplates developed by The Society for Biomolecular Screening (SBS) on behalf of and for acceptance by the American National Standards Institute.

The term “substantially” refers to an approximation. In certain embodiments, for example, a cell culture flask of the invention has dimensions that approximately correspond to the dimensions of a standard microplate.

The term “top” refers to the highest point, level, surface, or part of a device or system, or device or system component, when oriented for typical designed or intended operational use, such as positioning cell culture flasks for automated processing.

Cell Culture Flasks

While the present invention will be described with reference to a few specific embodiments, the description is illustrative of the invention and is not to be construed as limiting the invention. Various modifications can be made to the embodiments of the invention described herein by those skilled in the art without departing from the true scope of the invention as defined by the appended claims. It is also noted here that for a better understanding, certain like components are designated by like reference letters and/or numerals throughout the various figures.

The present invention provides cell culture flasks that can be utilized in a variety of automated processing applications, including introducing and removing fluids from the flasks using automated fluid handling systems. In many embodiments, for example, the cell culture flasks of the invention have dimensions that comply with SBS microplate standards and accordingly, are easily translocated by various types of robotic gripping mechanisms (e.g., without re-teaching travel paths) and can be positioned in or on devices that are designed to accommodate standard microtiter plates. Moreover, unlike many pre-existing flasks, the cell culture flasks of the invention can be accessed through top surfaces while the flasks are positioned in a horizontal position, e.g., in a nest of a positioning component of an automated system. Further, shorter fluid conveyance tips can typically be used to access a horizontally positioned flask than flasks that are configured to be processed in a vertical orientation. The longer tips used to exchange fluids in these vertically positioned flasks are generally harder to align than shorter tips. They also tend to bend and deflect upon entering these flask, which can damage the tips and flasks. Furthermore, the cell culture flasks described herein are compatible with disposable pipette tips which can be used to introduce fluids into or remove fluids from the cell culture flasks. The use of disposable tips greatly increases the throughput of processing systems which utilize this invention, and greatly reduces the risk of contamination and cross-contamination of the flasks, as there is no need to clean the tips between uses. Operating costs of systems utilizing these flasks can be similarly decreased. In addition, the cell culture flasks described herein may be single-use disposable flasks, which provide many of the advantages discussed with respect to the use of disposable tips, such as a drastic reduction in the risk of contamination or cross-contamination, and the elimination of the need to clean the individual flasks between uses. In addition, the configurations of the cell culture flasks of the invention generally provide more surface area for cells to adhere to than many pre-existing flasks. These and other features of the cell culture flasks in addition to related systems, components, and methods are described below.

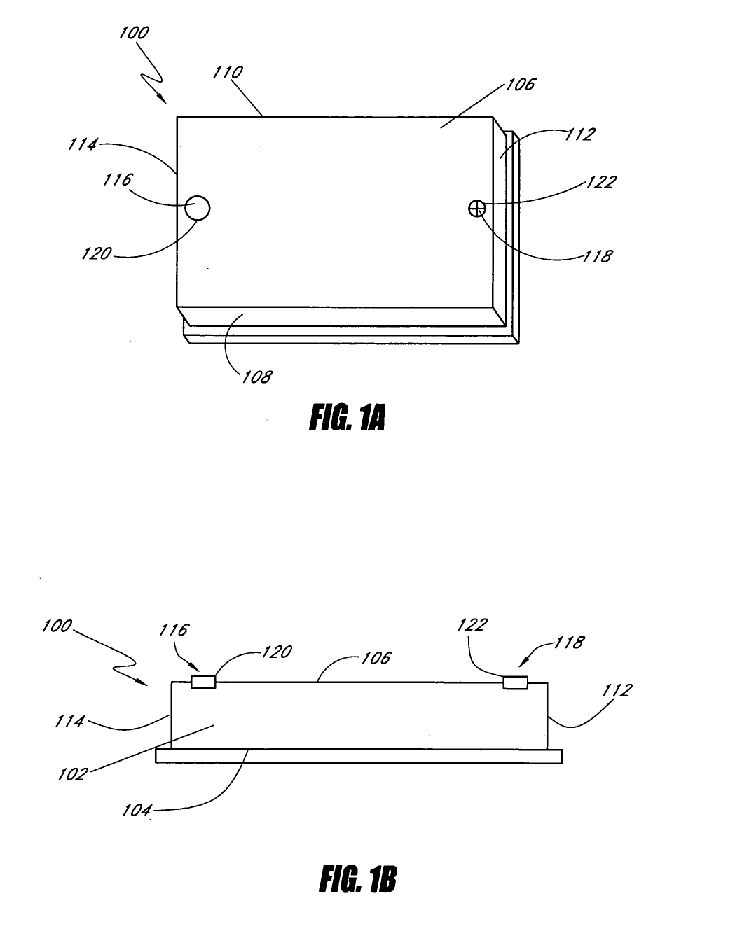

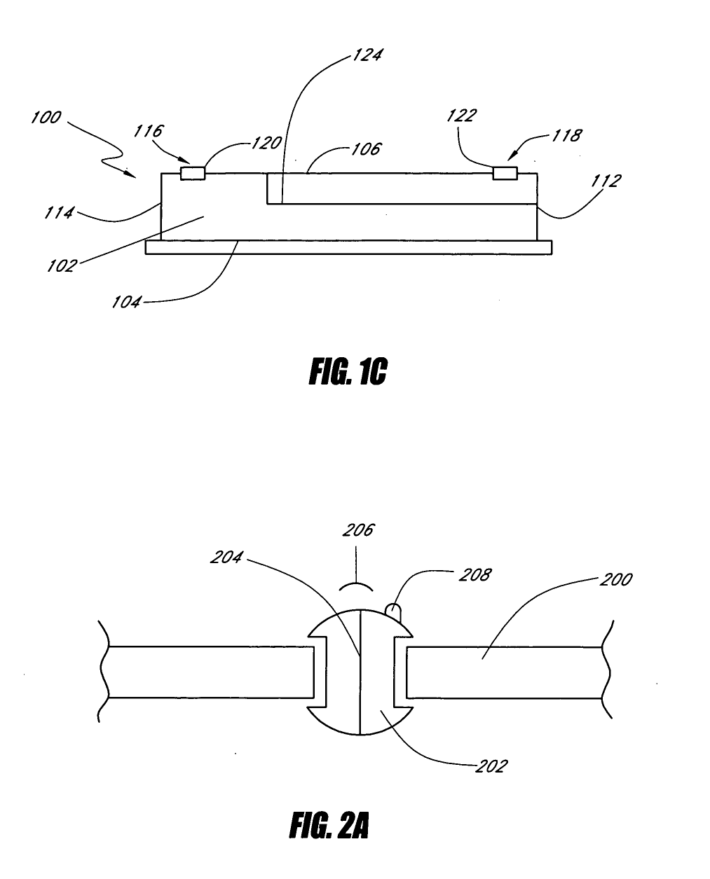

Referring initially to FIGS. 1A and B, cell culture flask 100 is schematically illustrated according to one embodiment of the invention. More specifically, FIG. 1A schematically shows cell culture flask 100 from a perspective view, while FIG. 1B schematically illustrates cell culture flask 100 from a side elevational view. As shown, cell culture flask 100 includes culture chamber 102 that is formed by bottom wall 104, top wall 106, first side 108 and second side wall 110. Second side wall 110 is opposite to first side wall 108. Cell culture flask 100 also includes first end wall 112 and second end wall 114 that is opposite to first end wall 112. In addition, cell culture flask 100 also includes vent opening 116 (including filter 120 disposed therein) in top wall 106 that allows air exchange between culture chamber 102 and an exterior of cell culture flask 100. To minimize the risk of contamination, vent openings are typically fitted with filters that block particle sizes of 0.22 μm or larger, although other filters are also optionally utilized. In some embodiments, vents include hydrophobic coatings to prevent fluid from wetting and clogging filters. Also included is access port opening 118 in top wall 106 through which fluids can be introduced into or removed from culture chamber 102. As shown, closure 122 (e.g., a lid, a septum, etc.) closes access port opening 118. Cell culture flask 100 is configured to allow introduction or removal of fluid through access port opening 118 into culture chamber 102 when cell culture flask 100 is positioned in a horizontal position, e.g., as shown in FIG. 1B. FIG. 1C schematically illustrates an embodiment of cell culture flask 100 that includes two tiers, one formed by bottom wall 104 and the other formed by shelf 124 disposed within culture chamber 102 of cell culture flask 100. Multiple tiers are typically utilized to increase the surface area within a given flask for growing cells. A septum is optionally substituted for filter 120 or included elsewhere in top wall 106.

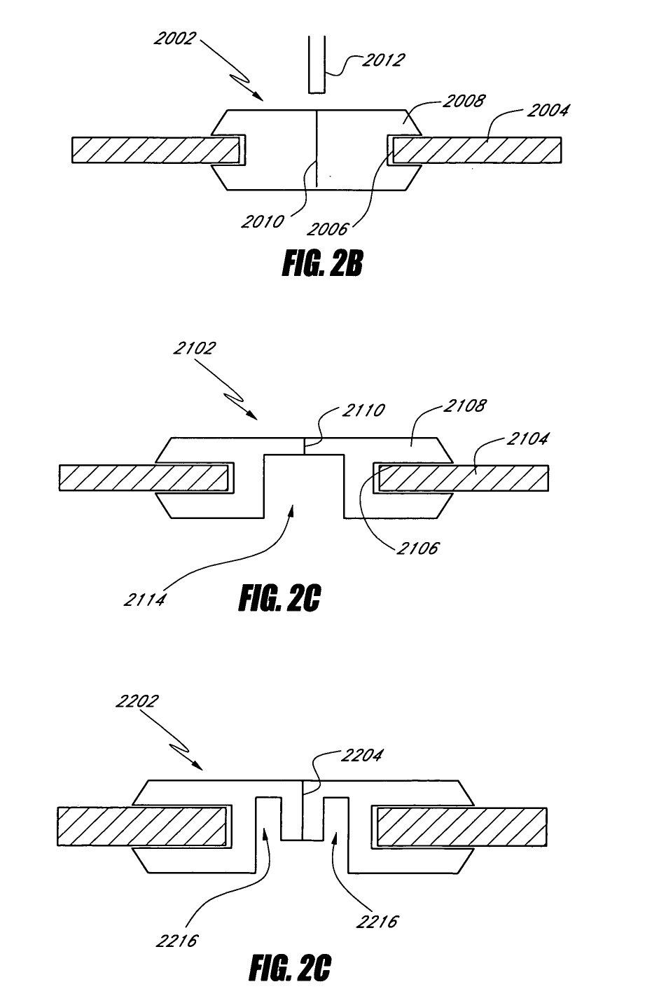

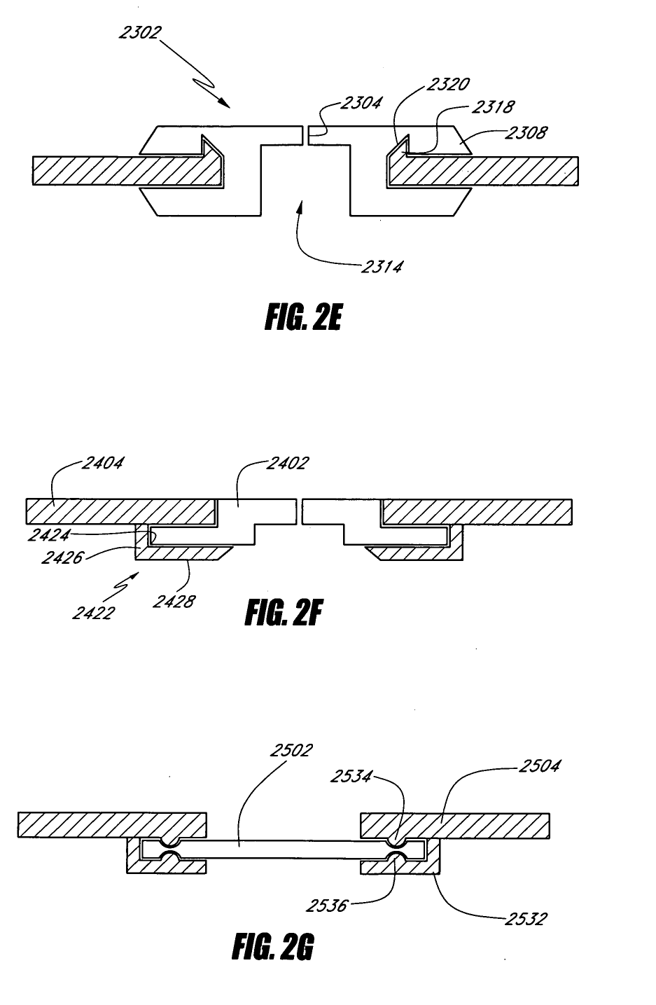

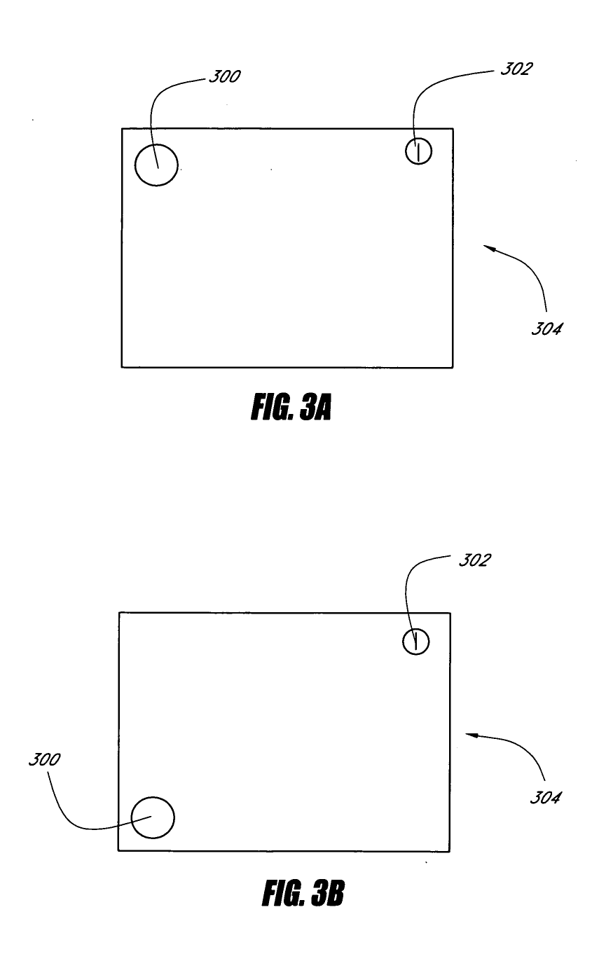

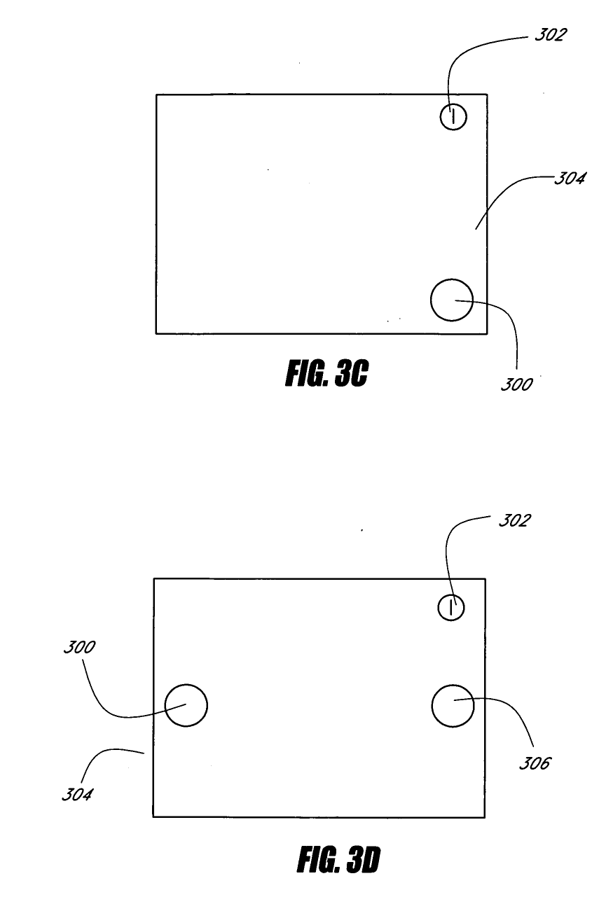

In some embodiments, cell culture flask closures (e.g., a lid, a septum, etc.) include contours that are shaped to direct fluid away from material exchange regions of the closures. To illustrate, FIG. 2 schematically depicts a segment of cell culture flask wall 200 having closure 202 disposed in an access port opening. As also shown, self-sealing channel 204 disposed through closure 202 in material exchange region 206 (i.e., closure 202 is pre-pierced). Pre-pierced closures allow access to flasks with blunt tips. Angled or tapered tips generally leave larger residual volumes than blunt tips, because angled tips can only maintain suction down to the top of the taper. Closures are typically pre-pierced in slot-shaped (see, e.g., FIG. 3A) or cross-shaped (see, e.g., FIG. 1A) configurations. Closure 202 has a rounded contour that acts to direct fluid droplet 208 away from material exchange region 206, e.g., to prevent fluid droplet 208 from being wicked through self-sealing channel 204, thereby minimizing the risk of contaminating the contents of the cell culture flask. In some embodiments, closures are fabricated from, or coated with, a hydrophobic material, such as polytetrafluoroethylene (TEFLON™) or the like to further facilitate directing fluids away from material exchange regions. Advantageously, because the cell culture flask closure is self-sealing, the access port opening need not be covered by a removable cover. Thus, the access port opening and the adjacent components need not comprise features configured to retain a removable cover in place, such as threading or snaps. The elimination of such complex retaining features facilitates automation of the introduction into or removal from the chamber of fluids, as there is no need to provide a robotic component capable of manipulating such removable closures.

FIG. 2B illustrates a cross-section of one embodiment of a septum 2002, such as the septum 118 of FIG. 1A. In the illustrated embodiment, the septum 2002 may be formed from a resilient material and secured in place relative to the flask wall 2004 via notch 2006 in the edges of the septum 2002. Advantageously, this notch 2006 extends around the perimeter of the septum 2002 such that a lip 2008 of the septum 2002 overlies a portion of the flask wall 2004, preventing fluid or other contaminants from passing in or out of the flask. The septum 2002 also includes a slit 2010 extending through the septum, such that a tip 2012 can be inserted through the slit. In further embodiments, multiple slits oriented at an angle to one another may extend through the septum 2002, as depicted with respect to the septum 118 of FIG. 1A. As discussed with respect to FIG. 2, in other embodiments, the septum 2002 (or other closure) may have a rounded profile.

Although the illustrated pre-pierced septum advantageously permits the use of a blunt tip to penetrate the septum, the use of a blunt tip may require the application of additional force to penetrate the septum. This force may result in the deformation of the septum, causing the septum to fold inwards, dislodging it from the flask wall. As this risks contamination of the material contained within the flask, in addition to necessitating manual intervention in what may be an otherwise automated process, it is desirable to reduce the risk that a septum may become dislodged. This can be accomplished at least by reducing the force required to penetrate the septum, or by better securing the septum to the flask wall, each of which are discussed in greater detail with respect to the embodiments below.

FIG. 2C illustrates another example of a septum 2102 which may be used with the various embodiments discussed above. The illustrated embodiment comprises a counterbore 2114 located underneath a slit 2110. Because the counterbore 2114 decreases the thickness, and therefore the stiffness, of the septum material through which the slit 2110 extends, the penetration force required to insert a blunt tip through the septum is decreased. It can be seen that in the illustrated embodiment, the reduction of the thickness of the septum material surrounding the slit 2110 is accomplished through the use of a counterbore 2114 having a substantially flat upper surface, but it will be understood that a cavity having an alternate shape, such as one having tapered edges or a tapered upper surface, may be used in place of the illustrated counterbore 2114. Advantageously, this counterbore 2114 or other cavity is provided on the underside of the septum 2102, facing the interior of the flask. While in other embodiments a cavity may be formed on the upper surface of the septum 2102, locating the cavity on the underside of the septum 2102 advantageously prevents pooling of fluid or other contaminants in the cavity prior to penetration, reducing the likelihood of contamination. In still further embodiments, a cavity may be located in both the upper and lower surfaces of the septum. In addition, the penetration force may be lowered by forming the septum 2102 from a material which has a low durometer, or hardness, reducing the force required to deform the material.

It can also be seen in FIG. 2C that the notch 2106 formed around the edge of the septum 2102 is deeper than in the septum 2002 of FIG. 2B. Thus, the notch 2106 engages a larger portion of the flask wall 2104, as the lips 2108 extending over the flask wall 2104 are longer. This further reduces the likelihood that the septum will fold up and be forced inside the cavity by the penetration force of a blunt tip.

FIG. 2D illustrates another embodiment of a septum 2202, similar to the septum 2102 of FIG. 2C. In this embodiment, rather than a counterbore formed directly beneath the slit 2204, an annular cavity 2216 is formed which extends around the slit 2204. It will be seen, however, that the thickness of the septum material through which the slit 2204 extends is greater than the thickness of the septum material over the annular cavity. Thus, the penetration force required to insert a tip through the slit 2204 is reduced due to the annular cavity 2216, but because the slit 2204 extends through a thicker portion of the septum, the slit 2204 is more likely to be resealed tightly upon removal of the tip from the slit 2204. In the illustrated embodiment, the thickness of the septum material surrounding the slit 2204 is less than the thickest portion of the septum 2212. However, in other embodiments, the thickness of the septum material surrounding the slot 2204 may be either equal to, or thicker than, the thickest portion of the septum 2202. In alternate embodiments, cavity 2216 may not be a continuous annular cavity, but may comprise two or more cavities 2216 spaced around the slit 2204. It will also be understood that the term annular need not refer to a structure which is substantially circular or ring-shaped, but may refer to any structure which circumscribes or extends about an interior region, and may be, for example, rectangular, triangular, trapezoidal, or any other desired shape.

FIG. 2E illustrates another embodiment of a septum 2302, in which the flask wall 2304 surrounding the septum 2302 comprises a feature configured to retain the septum 2302 in place during tip penetration. In the embodiment illustrated in FIG. 26, the feature configured to retain the septum is a barb. However, it will be appreciated that any mechanism for retaining the septum may be employed. The septum 2302 also comprises a counterbore 2314 located on the underside of the slit 2310. In the illustrated embodiment, a barb 2318 is located at or near the edge of the flask wall 2304, at the edge of the aperture through which the septum 2302 extends. In certain embodiments, the barb 2318 may comprise an annular barb extending around the edge of the aperture in the flask wall 2304. In other embodiments, two or more individual barbs 2318 may be positioned at various locations around the edge of the aperture in the flask wall 2304. A corresponding notch 2320 is formed in the lip 2308 of the septum 2302 extending over the barb 2318, and the barb 2318 engages the notch 2320. The barb 2318 serves to retain the septum 2302 in place during insertion of the tip, preventing the lip 2308 from being pulled toward the aperture in the flask wall 2304. In certain embodiments, the barb 2308 may be molded along with the rest of the flask wall 2304 at the time of manufacture. In other embodiments, the barb 2318 may be welded or otherwise secured to the flask wall 2304 at a later time.

FIG. 2F illustrates another embodiment of a septum 2402, in which a retaining feature is used to secure the septum in place. In the illustrated embodiment, the septum 2402 does not comprise a lip extending over the upper surface of flask wall 2404. Instead, retaining feature 2422 extends from the underside of the flask wall 2404, providing a notch 2424 configured to retain the edge of the septum 2402. In the illustrated embodiment, retaining feature 2422 comprises a first portion 2426 extending orthogonally downward from the flask wall 2404, and a second portion 2428 extending substantially parallel to the flask wall 2404, configured to inhibit movement of the septum away from the flask wall 2404. In one embodiment, retaining feature 2422 comprises a retaining ring extending about the aperture in the flask wall 2404, but in other embodiments, retaining feature 2422 comprises two or more individual retaining features extending downward from the underside of flask wall 2404. In FIG. 2F it can also be seen that the upper surface of the septum 2402 is advantageously either flush with the upper surface of the flask wall 2404, as illustrated, or extends above the upper surface of flask wall 2404, in order to prevent the pooling of fluid or other contaminants within the aperture in the flask wall.

FIG. 2G illustrates an embodiment of a septum 2502 and flask comprising a combination of the retention features discussed with respect to previous embodiments. In the illustrated embodiment, a retention ring 2532 extends from the underside of the flask wall 2504 to hold septum 2502 in place. The flask further comprises a ridge 2534 disposed on the underside of the flask wall 2504, and another ridge 2536 disposed on the interior surface of the retention ring 2532. In the illustrated embodiment, it can also be seen that the septum 2502 is provided with notches in the upper and lower surfaces of the septum corresponding to the ridges 2534 and 2536, such that the ridges 2534 and 2536 engage the notches. As discussed above, in one embodiment, the ridges 2534 and 2536 may comprise an annular structure extending about the apertures in the flask wall 2504 and the retention ring 2532, but in other embodiments the ridges 2534 and 2536 may comprise two or more distinct structures spaced about the apertures.

While specific embodiments of septums have been discussed with respect to the illustrated figures, it will be understood that other combinations of the above features are contemplated. It will be understood that any of the above features may be utilized in an embodiment either alone or in combination with one another, as each provide certain desirable benefits even in the absence of the other features.

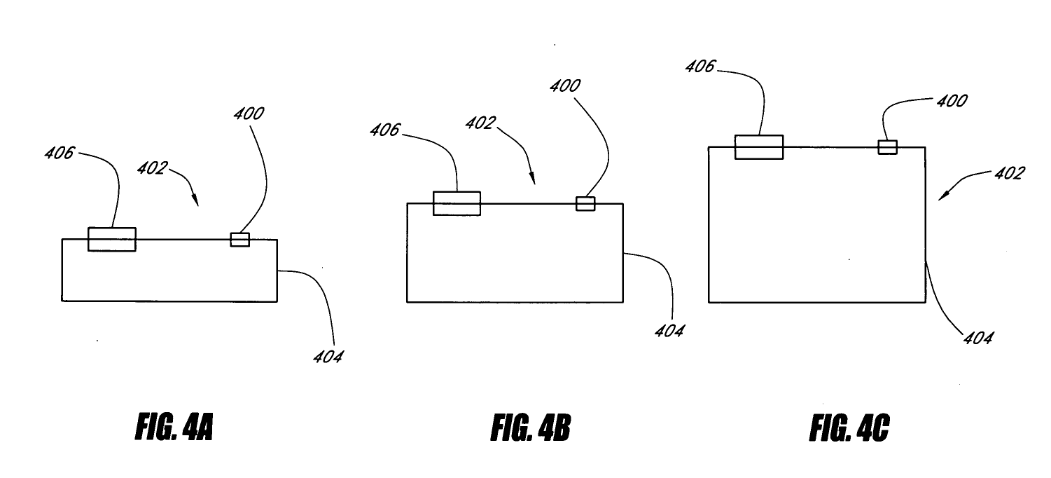

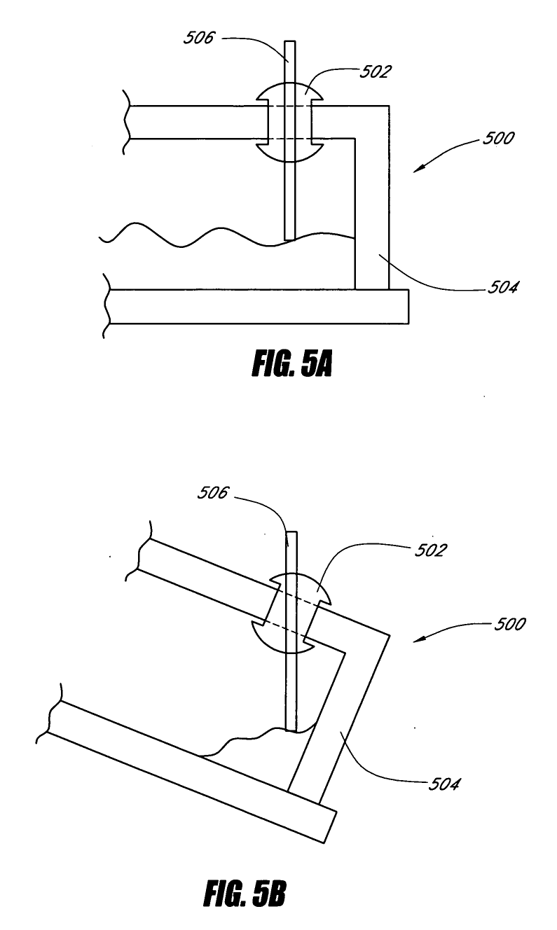

Essentially any configuration of vent and access port openings is optionally utilized. To illustrate, FIGS. 3A-C schematically show different exemplary configurations of vent opening 300 and access port opening 302 of cell culture flask 304. In certain embodiments, access port openings are disposed close to the edge of top walls, which permits multiple flasks to be stacked relative to one another for parallel processing applications. This aspect is described further below with respect to FIG. 13. In some embodiments, access port openings are placed at the same positional intervals as standard micro-well plates (e.g., 96-well plates, 384-well plates, etc.). This aspect provides additional compatibility with existing fluid dispensing devices or other micro-well plate processing systems. Vent and access port openings are also optionally disposed through other walls of the cell culture flasks aside from the top walls, e.g., through side walls and/or end walls. In addition, essentially any number of vent opening and/or access port opening is optionally included as desired for a given application. For example, FIG. 3D schematically illustrates an embodiment of cell culture flask 304 having two vent openings (300 and 306) in addition to access port opening 302. To further illustrate, FIGS. 4A-C schematically show cell culture flask embodiments having different volume capacities. As shown, septum 400 of cell culture flask 402 is spaced at a constant distance from side wall 404, e.g., so that the same fluid handling system can access cell culture flask 402 irrespective of varying volume capacities and spacing between septum 400 and filter 406. In addition, FIGS. 5A and B schematically illustrate cutaway cross-sectional views of cell culture flask 500, which includes septum 502 positioned proximal to side wall 504 to facilitate fluid removal from cell culture flask 500. As shown, fluid removal tip 506 can access and remove residual fluid from cell culture flask 500 when cell culture flask 500 is tilted towards side wall 504.

It will be understood that although certain of the illustrated embodiments of septums and other closures are depicted as being substantially symmetrical about the center, any suitable closure shape may be used. In one embodiment, the closure may comprise a septum substantially elongated in one direction. In a particular embodiment, depicted in FIG. 5C, a flask 508 comprises a septum 510 which is substantially rectangular in shape. Septum 510 comprises an aperture in the shape of a slot 512 aligned with the longer side of the septum 510. Advantageously, aperture 512 is oriented in a direction substantially orthogonal to an axis about which the flask pivots, such that entry of a tip at an angle not orthogonal to the upper surface of the flask is facilitated. In particular, when the flask 508 is oriented at an angle (as depicted in FIG. 5B, for example), a tip can still be inserted easily through the slot 512 due to the orientation of the slot.

The cell culture flasks of the invention optionally include cell concentration cavities or pockets disposed in a wall of the culture chambers. Cell concentration cavities concentrate cells from a fluid medium contained in the culture chambers when the cell culture flasks are subjected to a sufficient applied centrifugal force, e.g., using a conventional microplate centrifuge (e.g., with swinging buckets that transmit force down toward the bottom walls of horizontally positioned flasks) or the centrifuge systems described below in which force is transmitted toward the side walls of horizontally positioned flasks. Cells are concentrated in many different culturing applications including, for example, baculovirus production.

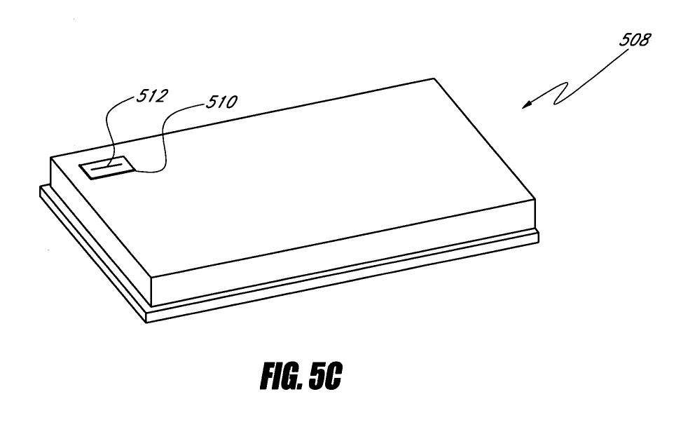

To illustrate, FIG. 6A schematically shows a cutaway perspective view of cell culture flask 600 that includes cell concentration cavity 602. FIG. 6B schematically illustrates cell culture flask 600 from a cross-sectional side view, whereas FIG. 6C schematically illustrates cell culture flask 600 from a top view. Walls 604 and 606 of cell culture flask 600 slope towards the cell concentration cavity to assist in directing or funneling cells into cell concentration cavity 602 under an applied centrifugal force. Additionally shown are filter 608, septum 610, and locational feature 612 (e.g., shown as an opaque surface region).

Locational feature 612 is positioned to trigger a position sensor (e.g., a laser sensor, etc.) of a storage device (e.g., a cell flask incubation device) when cell culture flask 600 is stored in the storage device. In certain embodiments, flasks are fabricated from clear materials. In these embodiments, light from a laser sensor may pass through such a flask and result in a false negative as to the presence of the flask in the absence of a locational feature. Optionally, vent openings (e.g., filters disposed therein), access port openings (e.g., septum or lids disposed therein), labels, and/or other features can also function as locational features (e.g., disrupt an incident laser beam from a sensor to register the presence of a flask in a storage device).

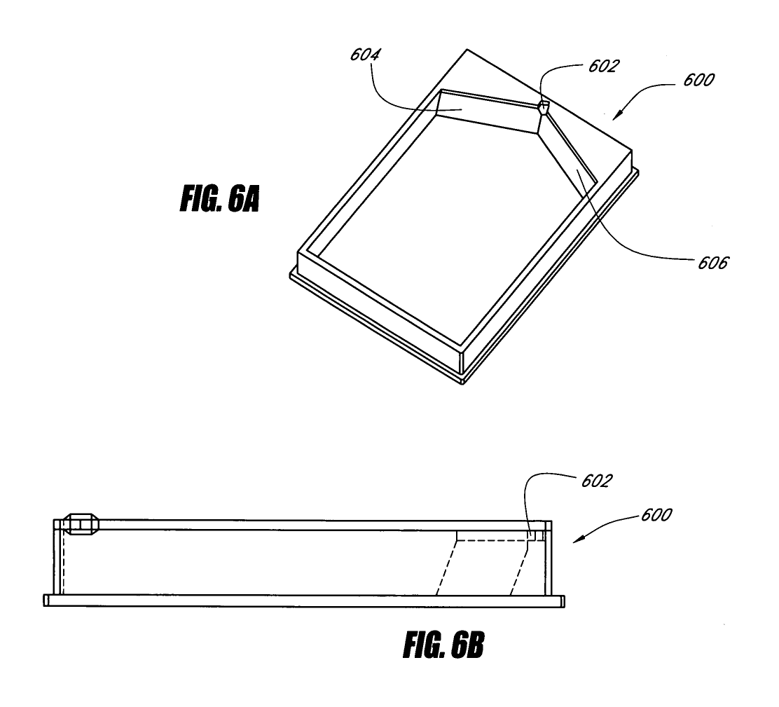

Typically, a cell concentration cavity is disposed in a wall of a culture chamber at a position such that the cell concentration cavity is above a selected fluid volume in the culture chamber, e.g., so that concentrated cells are not re-suspended in the fluid. To illustrate, FIG. 7 schematically shows a cross-sectional view of cell culture flask 700, which includes cell concentration cavity 702 positioned above fluid 704 disposed in horizontally positioned cell culture flask 700. Cell culture flask 600, described above, is another illustration of this aspect. The cell culture flask embodiments depicted in FIGS. 6 and 7 are well suited for use in the fixed nest centrifuge rotors, which are described below.

Cell concentration cavities typically have cross-sectional shapes selected from, e.g., a regular n-sided polygon, an irregular n-sided polygon, a triangle, a square, a rectangle, a trapezoid, a circle, an oval, and the like. In addition, these cavities are typically tapered at the bottom to allow for cell packing. When supernate is being withdrawn from flasks, cell concentration cavities protect the cells from being agitated into suspension.

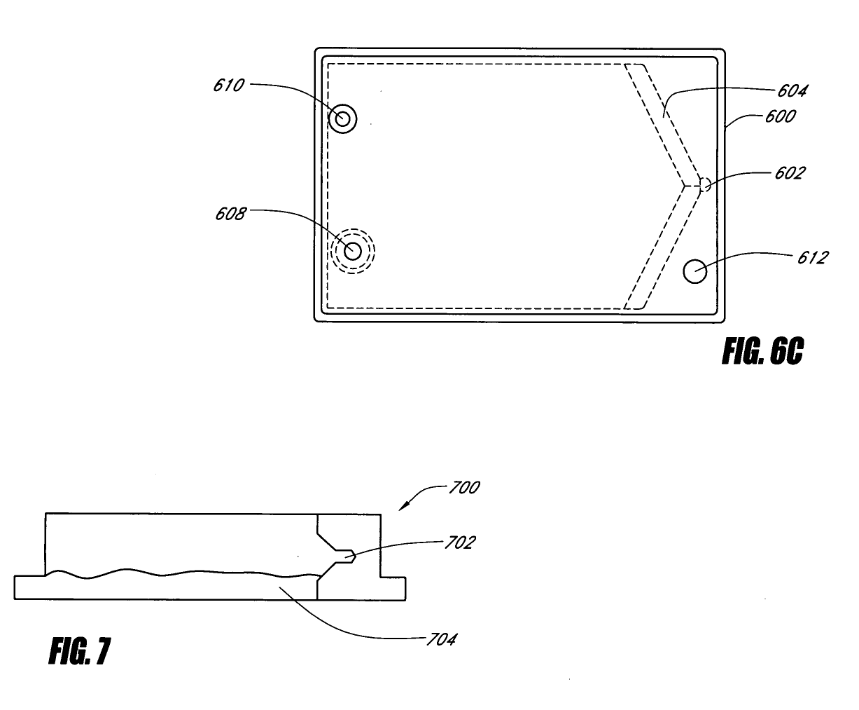

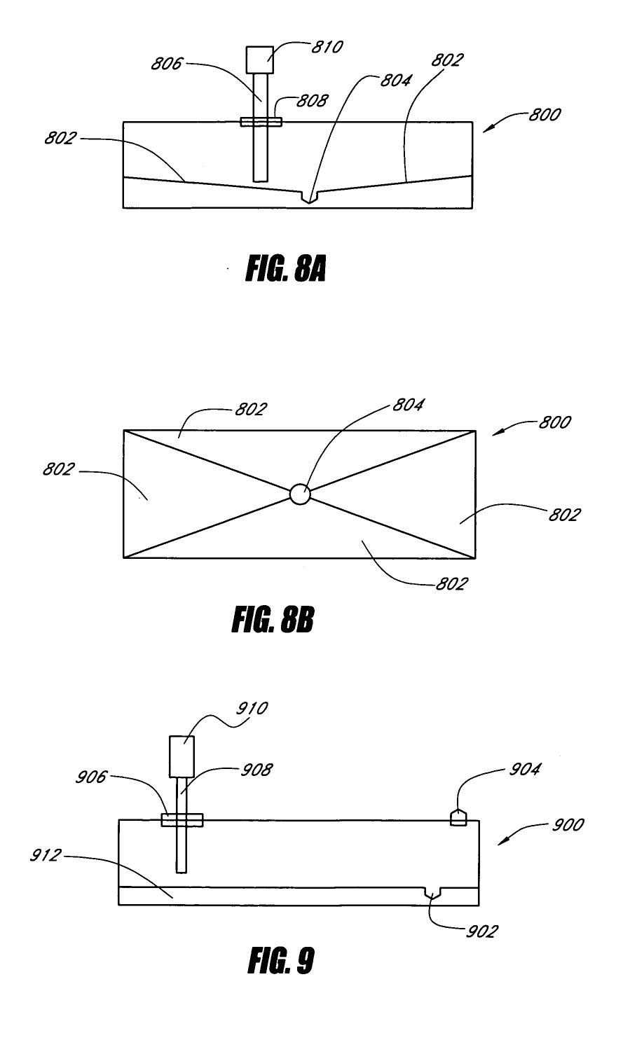

Additional examples of cell culture flasks that include cell concentration cavities are shown in FIGS. 8A, 8E, and 9. More specifically, FIG. 8A schematically shows cell culture flask 800 from a cross-sectional side view, whereas FIG. 8A schematically shows cell culture flask 800 from a cross-sectional top view. As shown, walls 802 slope or taper toward cell concentration cavity 804. Cell culture flask 800 also includes aspiration tip 806 disposed through septum 808. FIG. 9 schematically shows cell culture flask 900 from a cross-sectional side view in which cell concentration cavity 902 is disposed in bottom wall 912 of cell culture flask 900. Cell culture flask 900 also includes septa 904 and 906. Aspiration tip 908 is disposed through septum 906. A cell concentration cavity is typically positioned near the septum and the center of the flask when the bottom wall of the flask is tapered or sloped (see, e.g., FIG. 8A). This configuration tends to minimize the residual fluid volume of the supernate. When the bottom wall of the flask is substantially flat (see, e.g., FIG. 9), the septum is typically positioned away from the cell concentration cavity to minimize cell agitation when supernate is aspirated from the flask.

In some embodiments, the vent openings extend (e.g., form chimneys or the like) from an external surface of cell culture flasks, e.g., to minimize the wetting of filters disposed in the vent openings when the flasks are agitated. In these embodiments, the vent openings typically extend from the external surfaces at least about 5 mm, at least about 10 mm, at least about 25 mm, at least about 50 mm, or more. To illustrate, FIG. 10 schematically shows cell culture flask 1000 having vent opening 1004, which extends from external surface 1010 above fluid 1002. Vent opening 1004 includes filter 1006. In addition, cell culture flask 1000 also includes septum 1008.

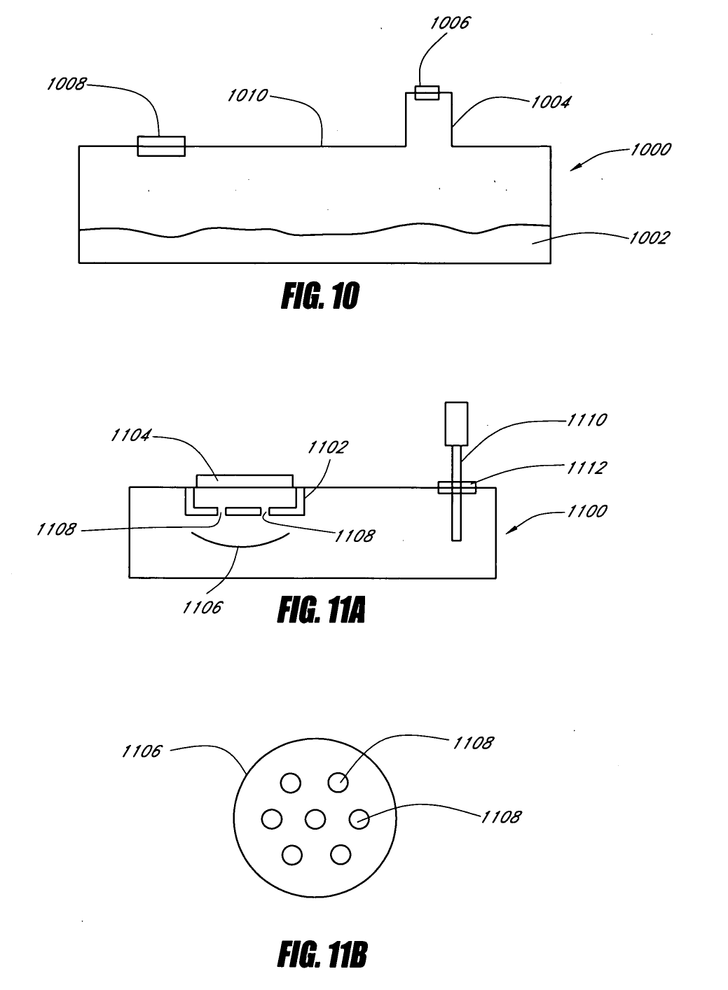

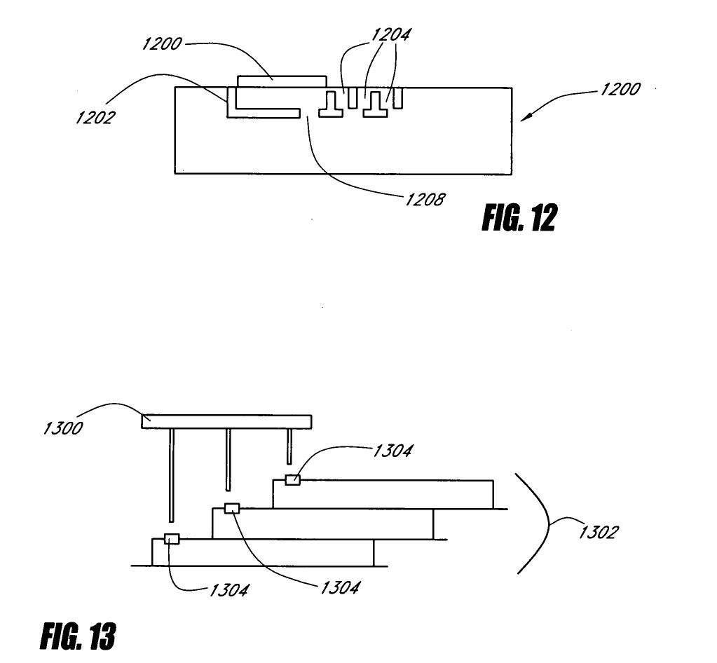

In certain embodiments, the walls of culture chambers include baffles that communicate with vent openings. Baffles are generally structured to prevent fluid from entering the vent opening when the fluid contacts the baffle, e.g., to prevent the vent from becoming clogged by a wetted filter. A clogged vent typically dead heads the flask, which can make it difficult for a pump to aspirate fluid from the flask and may induce error in aspirated volumes. To illustrate, FIG. 11A schematically shows a cross-sectional side view of cell culture flask 1100. As shown, cell culture flask 1100 includes baffle 1102 in communication with filter 1104 disposed in a vent opening. Baffle 1102 includes shield 1106 having holes 1108 disposed through shield 1106. Holes 1108 are typically sized such that surface tension of fluid causes one or more bubbles to form when the fluid contacts shield 1106 to prevent the fluid from entering the vent opening. Further, holes 1108 generally need to be large enough so that when fluid is aspirated from cell culture flask 100 through a tip, the pressure difference across the bubble will cause it to burst. FIG. 11B schematically illustrates a detailed bottom view of baffle 1102. Cell culture flask 1100 also includes tip 1110 disposed through septum 1112. To further illustrate, FIG. 12 schematically shows a cross-sectional side view of cell culture flask 1200. As shown, cell culture flask 1200 includes baffle 1202 that channels 1204 (e.g., a labyrinth of draining channels) that direct fluid away from filter 1206 disposed in the vent opening when the fluid enters baffle 1202 through hole 1208.

Automated Cell Culture Flask Processing Systems Components, and Applications

The invention also provides cell culture flask processing systems. In some embodiments, the systems include processing heads, cell culture flask positioning components that are structured to position cell culture flasks in a horizontal position, and translational mechanisms operably connected to the processing heads and/or the cell culture flask positioning components. The translational mechanisms are typically configured to move the processing heads and/or the cell culture flask positioning components relative to one another so that the processing heads can communicate with the horizontally positioned cell culture flasks. Typically, these cell culture flask processing systems also include controllers operably connected to the processing heads, the cell culture flask positioning components, and/or the translational mechanisms, e.g., to effect operations of these system components. These and other systems are described further below.

In some embodiments, for example, a processing head includes at least one tip. In these embodiments, the translational mechanism is typically configured to move the processing head and/or the cell culture flask positioning component relative to one another such that the tip accesses the cell culture flask through a septum when the cell culture flask is horizontally positioned on the cell culture flask positioning component. An example of this system configuration is provided below.

In certain embodiments, the processing head includes multiple tips that are configured to access multiple cell culture flasks when the cell culture flasks are stacked relative to one another on a cell culture flask positioning component (e.g., in a staggered, stair-like manner). In this approach, processing head tips can be closely spaced (i.e., have a small tip profile), thereby minimizing the axis travel utilized and hence, lowering costs. This is schematically illustrated in FIG. 13, which shows processing head 1300 positioned to access stacked cell culture flasks 1302 via septa 1304. In these embodiments, the cell culture flask processing system typically includes a fluid conveyance mechanism (e.g., a pump, etc.) operably connected to the tip (e.g., via a fluid conduit or the like). Fluid conveyance mechanisms are generally configured to introduce fluid into and/or remove fluid from the cell culture flask through the tip when the tip accesses the cell culture flask.

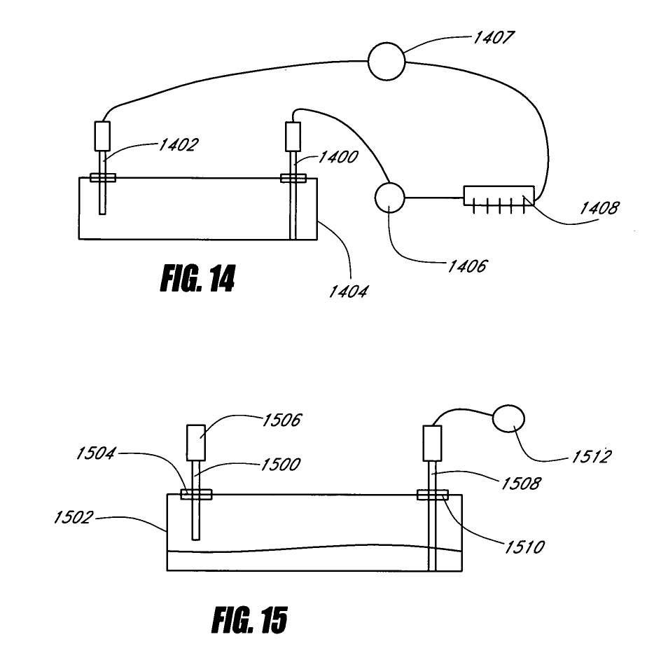

In some embodiments, the processing head includes two or more tips, and the fluid conveyance mechanism is configured to recirculate fluid disposed in the cell culture flask through the tips when the tips access the cell culture flask. To illustrate an embodiment, FIG. 14 schematically shows tips 1400 and 1402 in fluid communication with cell culture flask 1404. In the embodiment shown, tips 1400 and 1402 also fluidly communicate with fluid conveyance mechanisms 1406 and 1407 (e.g., peristaltic pumps, etc.) and dispensing head 1408. During a dispensing application, cell suspensions are typically recirculated through this fluid path, e.g., to mix fluid and prevent the cells from settling.

In another embodiment, tips are configured to allow gas exchange between cell culture flasks and exteriors of the flasks. As an illustration, FIG. 15 schematically shows venting tip 1500 disposed in cell culture flask 1502 via septum 1504. As also shown, filter 1506 is operably connected to venting tip 1500. Filter 1506 of venting tip 1500 prevents contamination. Aspirating tip 1508 is disposed in cell culture flask 1502 via septum 1510 and fluidly communicates with fluid conveyance mechanism 1512. Optionally, more than one venting tip (e.g., disposed through different septa of a cell culture flask) can be used during a given application in case one venting tip plugs with media.

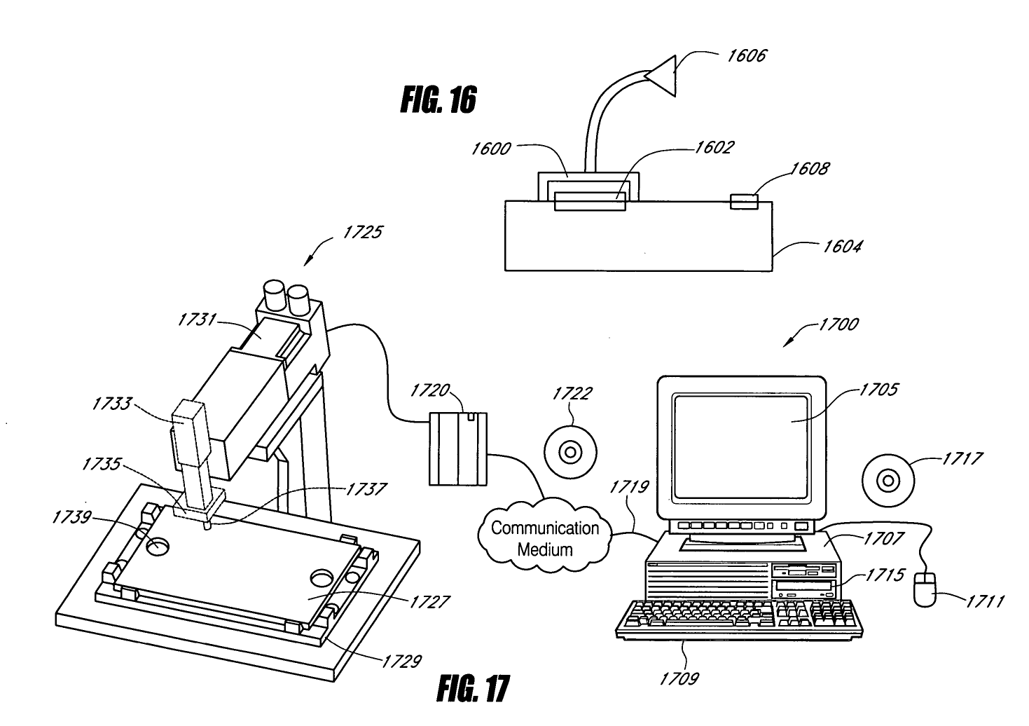

In certain embodiments, the processing head includes a pressure head (e.g., an external blow off unit) that is configured to put pressurized air (e.g., at about 1 psi) across a vent to displace collected fluids or media bubbles from the vent opening of a cell culture flask. Another advantage of this configuration is that the air from the pressure head is automatically filtered since it flows through the vent filter upon entering the flask. An embodiment of this configuration is schematically illustrated in FIG. 16, which shows pressure head 1600 communicating with vent opening 1602 of cell culture flask 1604. As further shown, pressure head 1600 is operably connected to pressure source 1606, which is configured to apply pressure to pressure head 1600 to effect the displacement of fluid from vent opening 1602 of cell culture flask 1604. Cell culture flask 1604 is accessible through septum 1608. In other embodiments, a pressure container or pressure cage can be used to provide pressurized air across a vent. Advantageously, the use of a device such as a pressure container can decrease the risk of damage, such as a rupturing of the flask, which may come about as a result of the use of a pressure head.

To further illustrate, FIG. 17 schematically depicts an exemplary cell culture flask processing system that includes an information appliance in which various aspects of the present invention may be embodied. As will be understood by practitioners in the art from the teachings provided herein, the invention is optionally implemented in hardware and software. In some embodiments, different aspects of the invention are implemented in either client-side logic or server-side logic. As will also be understood in the art, the invention or components thereof may be embodied in a media program component (e.g., a fixed media component) containing logic instructions and/or data that, when loaded into an appropriately configured computing device, cause that apparatus or system to perform according to the invention. As will additionally be understood in the art, a fixed media containing logic instructions may be delivered to a viewer on a fixed media for physically loading into a viewer's computer or a fixed media containing logic instructions may reside on a remote server that a viewer accesses through a communication medium in order to download a program component.

More specifically, FIG. 17 shows information appliance or digital device 1700 that may be understood as a logical apparatus (e.g., a computer, etc.) that can read instructions from media 1717 and/or network port 1719, which can optionally be connected to server 1720 having fixed media 1722. Information appliance 1700 can thereafter use those instructions to direct server or client logic, as understood in the art, to embody aspects of the invention. One type of logical apparatus that may embody the invention is a computer system as illustrated in 1700, containing CPU 1707, optional input devices 1709 and 1711, disk drives 1715 and optional monitor 1705. Fixed media 1717, or fixed media 1722 over port 1719, may be used to program such a system and may represent a disk-type optical or magnetic media, magnetic tape, solid state dynamic or static memory, or the like. In specific embodiments, the aspects of the invention may be embodied in whole or in part as software recorded on this fixed media. Communication port 1719 may also be used to initially receive instructions that are used to program such a system and may represent any type of communication connection. Optionally, aspects of the invention are embodied in whole or in part within the circuitry of an application specific integrated circuit (ACIS) or a programmable logic device (PLD). In such a case, aspects of the invention may be embodied in a computer understandable descriptor language, which may be used to create an ASIC, or PLD.

FIG. 17 also includes cell culture flask processing system 1725, which is operably connected to information appliance 1700 via server 1720. Optionally, cell culture flask processing system 1725 is directly connected to information appliance 1700. During a cell culture flask application, cell culture flask 1727 is typically placed in a horizontal position on cell culture flask positioning component 1729 (shown as a nest) by a robotic gripping apparatus (not shown). Robotic gripping apparatus are described further below. Translational mechanism. 1731 includes a Z-axis linear motion component (e.g., a solenoid motor), which moves processing head 1735 along the Z-axis. Although not shown, translational mechanism 1731 also includes an X/Y-axis linear motion component operably connected to cell culture flask positioning component 1729 to move cell culture flask 1727 into alignment relative to processing head 1735. Once cell culture flask 1727 is horizontally positioned on cell culture flask positioning component 1729, translational mechanism 1731 moves processing head 1735 so that tip 1737 pierces septum 1739 so that fluid can be introduced and/or removed fluid through tip 1737. Although not shown, tip 1737 is typically operably connected to a fluid conveyance mechanism (e.g., a pump, etc.) that effect fluid conveyance through tip 1737.

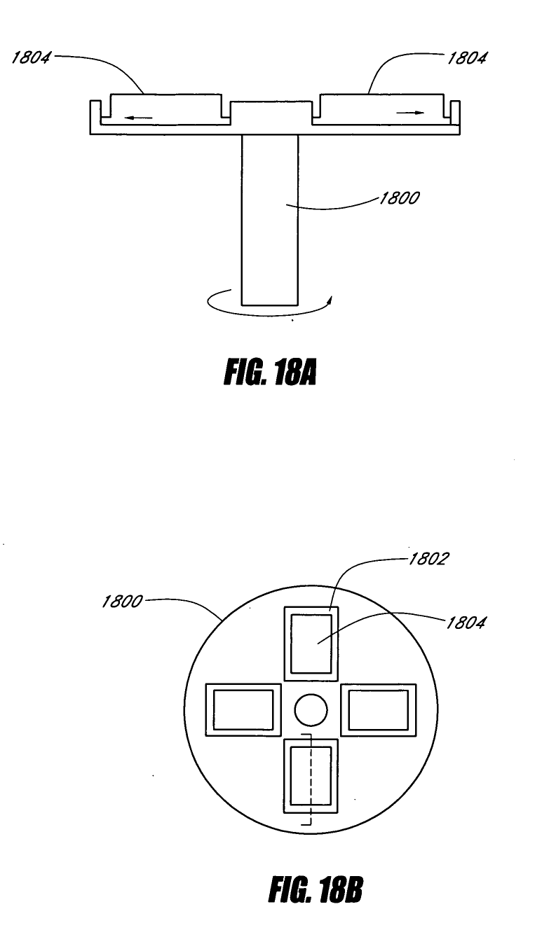

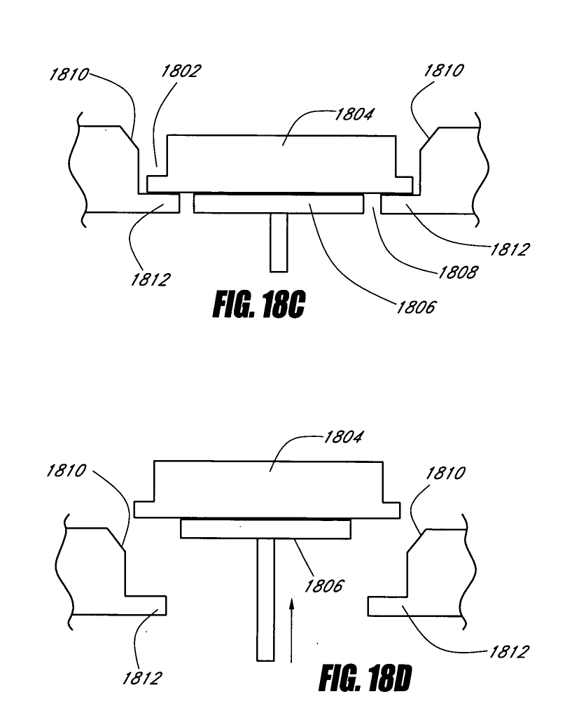

In another aspect, the invention provides centrifuge rotors (e.g., single piece rotors) that include fixed nests that are structured to receive cell culture flasks so that cells can be concentrated under an applied centrifugal force in, e.g., cell concentration cavities of the flasks. The single piece rotors are typically designed to transmit force to the sides of horizontally positioned plates. This is schematically illustrated in FIG. 18A, which depicts the rotation of cell culture flasks 1804 positioned in centrifuge rotor 1800. The arrows indicate the direction of the applied force. This rotor configuration assists in concentrating cells in cell concentration cavities of the flask embodiments depicted in, e.g., FIGS. 6A and 7, which are described further above.

These centrifuge rotors are generally configured to associate with lift mechanism that lower and raise cell culture flasks into and out of the nests, e.g., so that the flasks are accessible to robotic gripping devices, which translocate the flasks to and from the centrifuge rotors. FIG. 18B schematically shows a top view of centrifuge rotor 1800, which includes four fixed nests 1802 positioning cell culture flasks 1804. To further illustrate, FIG. 18C schematically illustrates a cross-sectional view of nest 1802 from centrifuge rotor 1800. As shown, orifice or cut out 1808 is disposed through nest 1802 to permit lift mechanism 1806 to lower and raise cell culture flask 1804 into and out of nest 1802. Lips 1812 are included to retain cell culture flask 1804 in position in nest 1802. FIG. 18D schematically illustrates cell culture flask 1804 raised by lift mechanism 1806 from nest 1802. Angled or chamfered surfaces 1810 of nest 1802 are included to facilitate the placement of cell culture flask 1804 in nest 1802.

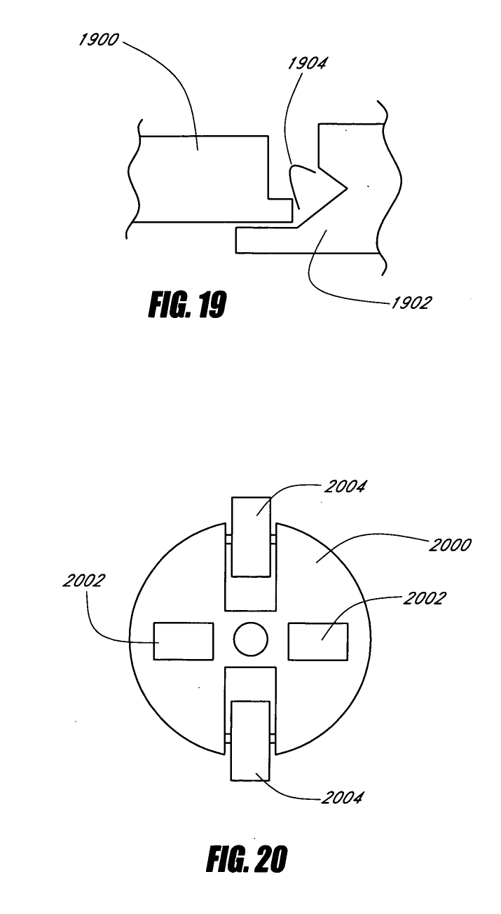

Centrifuge rotors typically include one or more retaining features that retain the cell culture flask when the centrifuge rotor rotates. An example of this aspect is schematically depicted in FIG. 19, which shows a detailed cross-sectional, cutaway view of cell culture flask 1900 placed in a nest of centrifuge rotor 1902. Retaining feature 1904 is shown as a notch fabricated into centrifuge rotor 1902. Retaining features such as these are generally fabricated into the walls of nests that are furthest away from the axis of rotation of the centrifuge rotor.

In some embodiments, centrifuge rotors include pivotal positioning components (e.g., in the form of a bucket or the like) that are receive cell culture flasks or other containers. To illustrate, FIG. 20 schematically shows a top view of centrifuge rotor 2000, which includes nests 2002 and pivotal positioning components 2004 that are each structured to receive cell culture flasks for centrifugation. As centrifuge rotor 2000 rotates, pivotal positioning components 2004 pivot away from the axis of rotation. Automated centrifuges that can be adapted for use with the centrifuge rotors of the invention are also described in, e.g., U.S. Patent Publication No. 200210132354, entitled “AUTOMATED CENTRIFUGE AND METHOD OF USING SAME,” filed Feb. 8, 2002 by Downs et al., which is incorporated by reference.

The controllers of the automated systems of the present invention are generally operably connected to and configured to control operation of system components, such as cell culture flask positioning components, translational mechanisms, fluid conveyance mechanisms, centrifuge rotors, lift mechanisms, etc. Controllers are generally included either as separate or integral system components that are utilized. Controllers and/or other system components is/are optionally coupled to an appropriately programmed processor, computer, digital device, or other logic device or information appliance (e.g., including an analog to digital or digital to analog converter as needed), which functions to instruct the operation of these instruments in accordance with preprogrammed or user input instructions (e.g., volumes to be conveyed, etc.), receive data and information from these instruments, and interpret, manipulate and report this information to the user.

A controller or computer optionally includes a monitor, which is often a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user. An exemplary system comprising a computer is schematically illustrated in FIG. 17.

The computer typically includes appropriate software for receiving user instructions, either in the form of user input into a set of parameter fields, e.g., in a GUI, or in the form of preprogrammed instructions, e.g., preprogrammed for a variety of different specific operations. The software then converts these instructions to appropriate language for instructing the operation of one or more controllers to carry out the desired operation, e.g., varying or selecting the rote or mode of movement of translational mechanisms, conveying fluids through conduits arid tips with pumps, or the like. The computer then receives the data from, e.g., sensors/detectors included within the system, and interprets the data, either provides it in a user understood format, or uses that data to initiate further controller instructions, in accordance with the programming, e.g., such as in monitoring detectable signal intensity, cell culture flask positioning, or the like: In some embodiments, a system of the invention includes a database that stores information about cell culture flasks, such as their location in the systems, flask contents, movement dates, etc. Cell culture flasks generally include bar codes or other labeling features that are read by bar code readers or the like to acquire or update this database information.

The computer can be, e.g., a PC (Intel x86 or Pentium chip-compatible DOS™, OS2™, WINDOWS™, WINDOWS NT™, WINDOWS95™, WINDOWS98™, WINDOWS2000™, WINDOWS XP™, LINUX-based machine, a MACINTOSH™, Power PC, or a UNIX-based (e.g., SUN™ work station) machine) or other common commercially available computer which is known to one of skill in the art. Standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Paradox™) can be adapted to the present invention. Software for performing, e.g., fluid conveyance, assay detection, and data deconvolution is optionally constructed by one of skill using a standard programming language such as AppleScript, Visual basic, C, C++, Perl, Python, Fortran, Basic, Java, or the like.

The automated systems of the invention are optionally further configured to detect and quantify absorbance, transmission, and/or emission (e.g., luminescence, fluorescence, etc.) of light, and/or changes in those properties in samples in or from the cell culture flasks described herein. Alternatively, or simultaneously, detectors can quantify any of a variety of other signals from cell culture flask samples including chemical signals (e.g., pH, ionic conditions, metabolites, dissolved oxygen, glucose, or the like), heat (e.g., for monitoring endothermic or exothermic reactions, e.g., using thermal sensors), or any other suitable physical phenomenon. In addition to other system components described herein, the systems of the invention optionally also include illumination or electromagnetic radiation sources, optical systems, and detectors. Because the systems and methods of the invention are flexible and allow various properties to be assayed, they can be used for all phases of assay development, including prototyping and mass screening.

Suitable signal detectors that are optionally utilized in these systems detect, e.g., emission, luminescence, transmission, fluorescence, phosphorescence, absorbance, or the like. In some embodiments, the detector monitors a plurality of optical signals, which correspond in position to “real time” results. Example detectors or sensors include PMTs, CCDs, intensified CCDs, photodiodes, avalanche photodiodes, optical sensors, scanning detectors, or the like. Each of these as well as other types of sensors is optionally readily incorporated into the systems described herein. The detector optionally moves relative to cell culture flasks or other sample containers, or alternatively, those containers move relative to the detector. Optionally, the systems of the present invention include multiple detectors. In these systems, such detectors are typically placed either in or adjacent to, e.g., cell culture flasks or other sample containers, such that the detector is in sensory communication with the cell culture flasks or other sample containers (i.e., the detector is capable of detecting the property of the sample for which that detector is intended).

The detector optionally includes or is operably linked to a computer, e.g., which has system software for converting detector signal information into assay result information or the like. For example, detectors optionally exist as separate units, or are integrated with controllers into a single instrument. Integration of these functions into a single unit facilitates connection of these instruments with the computer, by permitting the use of a few or even a single communication port for transmitting information between system components. Detection components that are optionally included in the systems of the invention are described further in, e.g., Skoog et al., Principles of Instrumental Analysis, 5^{th} Ed., Harcourt Brace College Publishers (1998) and Currell, Analytical Instrumentation: Performance Characteristics and Quality, John Wiley & Sons, Inc. (2000), which are both incorporated by reference.

The systems of the invention optionally also include at least one robotic translocation or gripping component that is structured to grip and translocate cell culture flasks between components of the automated systems and/or between the systems and other locations (e.g., other work stations, etc.). In certain embodiments, for example, systems further include gripping components that move cell culture flasks between positioning components, incubation or storage components, etc. Exemplary incubation devices that are optionally adapted for use with the systems of the present invention are described in, e.g., International Publication No. WO 03/008103, entitled “HIGH THROUGHPUT INCUBATION DEVICES,” filed Jul. 18, 2002 by Weselak et al., which is incorporated by reference. A variety of available robotic elements (robotic arms, movable platforms, etc.) can be used or modified for use with these systems, which robotic elements are typically operably connected to controllers that control their movement and other functions. Exemplary robotic gripping devices that are optionally adapted for use in the systems of the invention are described further in, e.g., U.S. Pat. No. 6,592,324, entitled “GRIPPER MECHANISM,” issued Jul. 15, 2003 to Downs et al., and International Publication No. WO 02/068157, entitled “GRIPPING MECHANISMS, APPARATUS, AND METHODS,” filed Feb. 26, 2002 by Downs et al., which are both incorporated by reference. Aspects of systems that are optionally adapted for use in the systems of the present invention are also described in, e.g., U.S. patent application Ser. No. 11/387,459, entitled “COMPOUND PROFILING DEVICE, SYSTEMS, AND RELATED METHODS,” filed Mar. 22, 2006 by Chang et al., which is incorporated by reference.

Fabrication Materials and Techniques

Cell culture flasks and components of the systems described herein are fabricated from materials or substrates that are generally selected according to properties, such as reaction inertness, durability, expense, or the like. In certain embodiments, for example, cell culture flasks are fabricated from various polymeric materials such as, polytetrafluoroethylene (TEFLON™), polypropylene, polystyrene, polysulfone, polyethylene, polymethylpentene, polydimethylsiloxane (PDMS), polycarbonate, polyvinylchloride (PVC), polymethylmethacrylate (PMMA), or the like. Polymeric parts are typically economical to fabricate, which affords cell culture flask disposability. Cell culture flasks or system components are also optionally fabricated from other materials including, e.g., glass, metal (e.g., stainless steel, anodized aluminum, etc.), silicon, or the like. For example, cell culture flasks are optionally assembled from a combination of materials permanently or removably joined or fitted together.

To further illustrate, the cell growth areas (e.g., bottom walls, etc.) of flasks are generally tissue culture treated to allow adherent cells to adhere to the flasks or to otherwise facilitate cell growth. Essentially any tissue culture coating can be utilized including, e.g., collagen, poly-D-lysine, poly-L-lysine, laminin, fibronectin, etc. These tissue culture treatments are generally restricted to designated growth areas only. For example, if the side or end walls of a flask are tissue culture treated, then some cells may adhere vertically before all of the cells settle to a growth surface on the bottom wall of a flask. The side and end walls are typically less than optimal for cell growth, because they are generally not completely submerged by media. This may lead to cell death, which tends to generate cellular debris that can be detrimental to, the health and growth of the remaining live cells. In certain embodiments, the surfaces of designated cells growth areas within flasks are fabricated to include various features that may facilitate the growth of certain types of cells. In some of these embodiments, for example, flask surfaces include ridges (e.g., in parallel lines, concentric circles, or other configurations) or other surfaces irregularities.

Cell culture flasks or system components are optionally formed by various fabrication techniques or combinations of such techniques including, e.g., injection molding, cast molding, machining, embossing, extrusion, etching, or other techniques. These and other suitable fabrication techniques are generally known in the art and described in, e.g., Rosato, Injection Molding Handbook, 3^{rd} Ed., Kluwer Academic Publishers (2000), Fundamentals of Injection Molding, W.J.T. Associates (2000), Whelan, Injection Molding of Thermoplastics Materials, Vol. 2, Chapman & Hall (1991), Fisher, Extrusion of Plastics, Halsted Press (1976), and Chung, Extrusion of Polymers: Theory and Practice, Hanser-Gardner Publications (2000). After cell culture flask or component part fabrication, the flasks or components are optionally further processed, e.g., by coating surfaces with, e.g., a hydrophilic coating, a hydrophobic coating, or the like.

Kits

The present invention also provides kits that include at least one cell culture flask or components thereof. The cell culture flasks of the kits of the invention are optionally pre-assembled (e.g., include components that are integral with one another, etc.) or unassembled. In addition, kits typically further include appropriate instructions for assembling, utilizing, and maintaining the cell culture flasks or components thereof. Kits also typically include packaging materials or containers for bolding kit components.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A cell culture flask, comprising:
a culture chamber that is formed by a bottom wall, a top wall, a first side wall and a second side wall that is opposite to the first side wall, a first end wall and a second end wall that is opposite to the first end wall;
a vent opening in the top wall that allows air exchange between the culture chamber and an exterior of the flask; and,
an access port opening in the top wall through which fluids can be introduced into or removed from the culture chamber;
wherein the cell culture flask is configured to allow introduction or removal of fluid through the access port opening into the culture chamber when the cell culture flask is positioned in a horizontal position.

2. The cell culture flask of claim 1, comprising at least one labeling feature.

3. The cell culture flask of claim 1, comprising at least one tier formed within the culture chamber.

4. The cell culture flask of claim 1, comprising one or more dimensions that substantially correspond to one or more dimensions of a standard multi-well plate.

5. The cell culture flask of claim 1, wherein the vent opening comprises a filter.

6. The cell culture flask of claim 1, comprising at least one cell concentration cavity disposed in at least one wall of the culture chamber, which cell concentration cavity is structured to concentrate cells from a fluid medium contained in the culture chamber when the cell culture flask is subjected to a sufficient applied centrifugal force.

7. The cell culture flask of claim 6, wherein the cell concentration cavity has a cross-sectional shape selected from the group consisting of: a regular n-sided polygon, an irregular n-sided polygon, a triangle, a square, a rectangle, a trapezoid, a circle, and an oval.

8. The cell culture flask of claim 6, wherein one or more walls of the culture chamber slope towards the cell concentration cavity.

9. The cell culture flask of claim 6, wherein cell concentration cavity is disposed in the wall of the culture chamber at a position such that the cell concentration cavity is above a selected fluid volume when the selected fluid volume is contained in the culture chamber.

10. The cell culture flask of claim 1, comprising at least one locational feature that is positioned to trigger a sensor of a storage device when the cell culture flask is stored in the storage device.

11. The cell culture flask of claim 10, wherein the locational feature comprises the vent opening and/or the access port opening.

12. The cell culture flask of claim 1, wherein the top wall comprises a baffle that communicates with the vent opening, which baffle is structured to prevent fluid from entering the vent opening when the fluid contacts the baffle.

13. The cell culture flask of claim 12, wherein the baffle comprises a shield having one or more holes disposed through the shield, which holes are sized such that surface tension of the fluid causes one or more bubbles to form when the fluid contact the shield to prevent fluid from entering the vent opening.

14. The cell culture flask of claim 12, wherein the baffle comprises one or more channels that direct the fluid away from the vent opening when the fluid enters the baffle.

15. The cell culture flask of claim 1, comprising a closure that closes the access port opening.

16. The cell culture flask of claim 15, wherein the closure comprises a septum or a lid.

17. The cell culture flask of claim 15, wherein the closure comprises a material exchange region and wherein a contour of the closure is shaped to direct fluid away from the material exchange region when the fluid contacts the material exchange region.

18. The cell culture flask of claim 17, wherein the contour of the closure disposed proximal to the material exchange region is rounded.

19. The cell culture flask of claim 17, wherein the material exchange region comprises a self-sealing channel disposed through the closure.

20. The cell culture flask of claim 1, where the access port opening does not comprise features configured to retain a removable cover.

21. The cell culture flask of claim 1, wherein the vent opening extends from an external surface of the cell culture flask.

22. The cell culture flask of claim 21, wherein the vent extends at least 10 mm from the external surface of the cell culture flask.

23. A cell culture flask, comprising:
a culture chamber that is formed by a bottom wall, a top wall, a first side wall and a second side wall that is opposite to the first side wall, a first end wall and a second end wall that is opposite to the first end wall; and
a cell concentration cavity disposed in at least one wall of the culture chamber, which cell concentration cavity is structured to concentrate cells from a fluid medium contained in the culture chamber when the cell culture flask is subjected to a sufficient applied centrifugal force.

24. The cell culture flask of claim 23, wherein the cell concentration cavity has a cross-sectional shape selected from the group consisting of: a regular n-sided polygon, an irregular n-sided polygon, a triangle, a square, a rectangle, a trapezoid, a circle, and an oval.

25. The cell culture flask of claim 23, wherein one or more walls of the culture chamber slope towards the cell concentration cavity.

26. The cell culture flask of claim 23, wherein cell concentration cavity is disposed in the wall of the culture chamber at a position such that the cell concentration cavity is above a selected fluid volume when the selected fluid volume is contained in the culture chamber.

27. A cell culture flask, comprising:
a culture chamber that is formed by a bottom wall, a top wall, a first side wall and a second side wall that is opposite to the first side wall, a first end wall and a second end wall that is opposite to the first end wall;
a vent opening in at least one wall of the culture chamber that allows air exchange between the culture chamber and the exterior of the flask; and,
a baffle that communicates with the vent opening, which baffle is structured to prevent fluid from entering the vent opening when the fluid contacts the baffle.

28. The cell culture flask of claim 27, wherein the baffle comprises a shield having one or more holes disposed through the shield, which holes are sized such that surface tension of the fluid causes one or more bubbles to form when the fluid contact the shield to prevent fluid from entering the vent opening.

29. The cell culture flask of claim 27, wherein the baffle comprises one or more channels that direct the fluid away from the vent opening when the fluid enters the baffle.

30. A cell culture flask comprising:
a culture chamber that is formed by a bottom wall, a top wall, a first side wall and a second side wall that is opposite to the first side wall, a first end wall and a second end wall that is opposite to the first end wall;
an access port opening in at least one wall of the culture chamber through which fluids can be introduced into or removed from the culture chamber; and,
a closure that closes the access port opening, which closure comprises a material exchange region.

31. The cell culture flask of claim 30, wherein a contour of the closure is shaped to direct fluid away from the material exchange region when the fluid contacts the material exchange region.

32. The cell culture flask of claim 30, wherein the closure comprises a septum or a lid.

33. The cell culture flask of claim 30, wherein the contour of the closure disposed proximal to the material exchange region is rounded.

34. The cell culture flask of claim 30, wherein the material exchange region comprises a self-sealing channel disposed through the closure.

35. The cell culture flask of claim 30, wherein the at least one wall of the culture chamber through which the access port opening extends comprises a closure retention feature.

36. The cell culture flask of claim 35, wherein the closure comprises a septum.

37. The cell culture flask of claim 35, wherein the retention feature is located on an interior side of the wall through which the access port opening extends, and comprises a notch configured to retain a portion of the closure.

38. The cell culture flask of claim 37, wherein the retention feature comprises an annular structure extending around the periphery of the access port opening.

39. The cell culture flask of claim 38, wherein a ridge extends from an upper surface of the retention feature.

40. The cell culture flask of claim 39, wherein the ridge comprises an annular ridge.

41. The cell culture flask of claim 38, wherein a ridge extends from an interior surface of the wall through which the access port opening extends, wherein the ridge is positioned opposite the retention feature.

42. The cell culture flask of claim 41, wherein the ridge comprises an annular ridge extending about the access port opening.

43. The cell culture flask of claim 37, additionally comprising at least one additional retention feature located on the interior side of the wall through which the access port opening extends.

44. The cell culture flask of claim 35, wherein the retention feature comprises a barb extending from an exterior side of the wall through which the access port opening extends.

45. The cell culture flask of claim 44, wherein the closure comprises a notch, and wherein the barb is configured to engage the notch.

46. The cell culture flask of claim 44, wherein the barb comprises an annular structure extending about the periphery of the access port opening.

47. The cell culture flask of claim 44, additionally comprising at least one additional barb located on the exterior side of the wall through which the access port opening extends.

48. The cell culture flask of claim 44, wherein a thickness of the closure varies.

49. The cell culture flask of claim 48, wherein the closure comprises a septum, and wherein the material exchange region comprises an aperture extending through the septum.

50. The cell culture flask of claim 49, wherein the aperture extending through the septum comprises at least one slit.

51. The cell culture flask of claim 49, wherein the septum comprises a cavity formed on the underside of the septum.

52. The cell culture flask of claim 51, wherein the cavity is located underneath the aperture extending through the septum.

53. The cell culture flask of claim 52, wherein the cavity comprises a counterbore.

54. The cell culture flask of claim 52, wherein the cavity comprises an exterior annular portion, and an interior portion through which the aperture extends wherein a height of the cavity is less than the height of the cavity in the exterior annular portion.

55. The cell culture flask of claim 51, wherein the cavity is located away from the aperture extending through the septum.

56. The cell culture flask of claim 55, wherein the cavity comprises an annular portion extending about a portion of the septum through which the aperture extends.

57. The cell culture flask of claim 55, additionally comprising at least one additional cavity located away from the aperture extending though the septum.

58. The cell culture flask of claim 30, wherein the closure comprises a septum having a shape which is elongated in a first direction and wherein the material exchange region comprises a slot oriented in a direction substantially the same as the first direction.

59. The cell culture flask of claim 58, wherein the septum is substantially rectangular in shape, and wherein the slot is oriented substantially parallel to a long side of the septum.

60. A cell culture flask, comprising:
a culture chamber that is formed by a bottom wall, a top wall, a first side wall and a second side wall that is opposite to the first side wall, a first end wall and a second end wall that is opposite to the first end wall; and,
a vent opening in at least one wall of the culture chamber and extending from an external surface of the cell culture flask, which vent opening allows air exchange between the culture chamber and an exterior of the flask.

61. The cell culture flask of claim 60, wherein the vent opening comprises a filter.

62. The cell culture flask of claim 60, wherein the vent opening extends at least 10 mm from the external surface of the cell culture flask.

63. A container closure, comprising a material exchange region, wherein a contour of the container closure is shaped to direct fluid away from the material exchange region when the fluid contacts the material exchange region.

64. The container closure of claim 63, wherein the contour of the closure disposed proximal to the material exchange region is rounded.

65. The container closure of claim 63, wherein the material exchange region comprises a self-sealing channel disposed through the closure.

66. A cell culture flask processing system, comprising:
a processing head;
a cell culture flask positioning component that is structured to position at least one cell culture flask in a horizontal position; and,
a translational mechanism operably connected to the processing head and/or the cell culture flask positioning component, which translational mechanism is configured to move the processing head and/or the cell culture flask positioning component relative to one another such that the processing head communicates with the cell culture flask when the cell culture flask positioning component positions the cell culture flask in the horizontal position.

67. The cell culture flask processing system of claim 66, comprising a controller operably connected to the processing head, the cell culture flask positioning component, and/or the translational mechanism.

68. The cell culture flask processing system of claim 66, wherein the processing head comprises at least one tip, and wherein translational mechanism is configured to move the processing head and/or the cell culture flask positioning component relative to one another such that the tip accesses the cell culture flask when the cell culture flask positioning component positions the cell culture flask in a horizontal position.

69. The cell culture flask processing system of claim 68, wherein the processing head comprises multiple tips that are configured to access multiple cell culture flasks when the cell culture flasks are stacked relative to one another on the cell culture flask positioning component.

70. The cell culture flask processing system of claim 68, comprising a fluid conveyance mechanism operably connected to the tip, which fluid conveyance mechanism is configured to introduce fluid into and/or remove fluid from the cell culture flask through the tip when the tip accesses the cell culture flask.

71. The cell culture flask processing system of claim 70, wherein the processing head comprises at least two tips, and wherein the fluid conveyance mechanism is configured to recirculate fluid disposed in the cell culture flask through the tips when the tips access the cell culture flask.

72. The cell culture flask processing system of claim 68, wherein the tip is configured to allow gas exchange between the cell culture flask and an exterior of the cell culture flask when the tip accesses the cell culture flask.

73. The cell culture flask processing system of claim 68, wherein a filter is operably connected to the tip.

74. The cell culture flask processing system of claim 66, wherein the processing head comprises a pressure head configured to communicate with a vent opening of the cell culture flask.

75. The cell culture flask processing system of claim 74, comprising a pressure source operably connected to the pressure head, which pressure source is configured to apply pressure to the pressure head when the pressure head communicates with the vent opening of the cell culture flask to effect displacement of fluid from the vent opening of the cell culture flask.

76. A centrifuge rotor, comprising at least one nest that is structured to receive a cell culture flask, which centrifuge rotor is structured to rotate the cell culture flask in a horizontal position so that cells in a cell suspension contained in the cell culture flask collect on a side wall of the cell culture flask.

77. The centrifuge rotor of claim 76, wherein a position of the nest is fixed in the centrifuge rotor.

78. The centrifuge rotor of claim 76, comprising one or more pivotal positioning components that are structured to receive the cell culture flask or another container, which pivotal positioning components pivot when the centrifuge rotor rotates.

79. The centrifuge rotor of claim 76, wherein the nest comprises one or more angled surfaces that direct the cell culture flask into the nest when the nest receives the cell culture flask.

80. The centrifuge rotor of claim 76, wherein the nest comprises one or more retaining features that retain the cell culture flask when the centrifuge rotor rotates.

81. A centrifuge system comprising the centrifuge rotor of claim 76.

82. The centrifuge rotor of claim 76, wherein the nest is configured to associate with a lift mechanism such that the lift mechanism can raise and/or lower the cell culture flask when the cell culture flask is present in the nest and the centrifuge rotor is at rest.

83. The centrifuge rotor of claim 82, comprising an orifice disposed through the nest, which orifice allows the lift mechanism to raise and/or lower the cell culture flask when the cell culture flask is present in the nest and the centrifuge rotor is at rest.

84. A centrifuge rotor, comprising at least one nest that is structured to receive a cell culture flask, which nest is configured to associate with a lift mechanism such that the lift mechanism can raise and/or lower the cell culture flask when the cell culture flask is present in the nest and the centrifuge rotor is at rest.

85. The centrifuge rotor of claim 84, wherein a position of the nest is fixed in the centrifuge rotor.

86. The centrifuge rotor of claim 84, comprising an orifice disposed through the nest, which orifice allows the lift mechanism to raise and/or lower the cell culture flask when the cell culture flask is present in the nest.

87. The centrifuge rotor of claim 84, comprising one or more pivotal positioning components that are structured to receive the cell culture flask or another container, which pivotal positioning components pivot when the centrifuge rotor rotates.

88. The centrifuge rotor of claim 84, wherein the nest comprises one or more angled surfaces that direct the cell culture flask into the nest when the nest receives the cell culture flask.

89. The centrifuge rotor of claim 84, wherein the nest comprises one or more retaining features that retain the cell culture flask when the centrifuge rotor rotates.

90. A centrifuge system comprising the centrifuge rotor of claim 84.

91. A method of processing a cell culture flask, the method comprising:
positioning the cell culture flask in a horizontal position; and,
introducing and/or removing fluid through an access port opening disposed in a top wall of the cell culture flask, thereby processing the cell culture flask.

92. A method of concentrating cells in a cell culture flask, the method comprising:
placing a cell culture flask containing a cell suspension into a centrifuge rotor; and,
rotating the cell culture flask in a horizontal position in the centrifuge rotor at a rate that is sufficient to cause cells in the cell suspension to collect on a side wall of the cell culture flask, thereby concentrating the cells in the cell culture flask.
